(19) United States
(12) Patent Application Publication
(10) Pub. No.: US 2022/0192769 A1
(43) Pub. Date: Jun. 23, 2022

(54) SURGICAL INSTRUMENT AND MEDICAL MANUPULATOR SYSTEM

(71) Applicant: SONY GROUP CORPORATION, TOKYO (JP)

(72) Inventors: WILLIAM ALEXANDRE CONUS, TOKYO (JP); MASAYA KINOSHITA, KANAGAWA (JP); YASUHISA KAMIKAWA, TOKYO (JP); TAKUYA KOMAMI, TOKYO (JP)

(21) Appl. No.: 17/593,268

(22) PCT Filed: Feb. 6, 2020
(86) PCT No.: PCT/JP2020/004498

§ 371 Date: Sep. 14, 2021

(30) Foreign Application Priority Data
Mar. 26, 2019 (JP) 2019-057712

(51) Int. Cl. A61B 34/37; A61B 17/068; A61B 17/295; B25J 15/04; B25J 3/00

(52) U.S. Cl. CPC A61B 34/37; A61B 17/068; B25J 3/00; B25J 15/04; A61B 17/295

(57) ABSTRACT

There is provided a surgical instrument having a plurality of methods of use and reducing the number of times that treatment portions are detachably replaced. The surgical instrument includes a treatment unit having a first treatment portion on an end portion thereof and a second treatment portion on another end portion thereof, and a holder that supports the treatment unit so as to allow the states of use of the first treatment portion and the second treatment portion to be switched. The holder supports the holder so as to be rotatable around a switching shaft for switching the states of use of the first treatment portion and the second treatment portion, and supports the treatment unit such that, while one of the first treatment portion and the second treatment portion is in a usable state, the other is in a standby state.

### TECHNICAL FIELD

The technology disclosed in the present description relates to a surgical instrument for use in a medical manipulator system and also to a medical manipulator system on which one or more surgical instruments are mounted.

### BACKGROUND ART

In recent years, robotics is making so much progress that it is finding widespread use in various industrial sites. For example, a master-slave robot system allows a person (operator) to remotely control a manipulator when the operator operates a master arm at hand and a remote slave arm traces the motion of the master arm. Master-slave robot systems, such as surgical robots, are used in industrial fields where computer-controlled fully autonomous actions are still hard to achieve.

Surgical robots equipped with a plurality of surgical instruments including forceps are also known in the art. Basically, surgical instruments can be detached from arms that hold the surgical instruments, and surgical robots can change and use many types of surgical instruments more than those which can be mounted on the surgical robots at a time.

Further, surgical instruments need to be sterilized and cleaned each time they are used. Therefore, there have been proposed surgical instruments with treatment tools detachably mounted thereon (see, for example, PTL 1).

### CITATION LIST

### Patent Literature

### [PTL 1]

PCT Patent Publication No. WO2013/018897

### SUMMARY

### Technical Problem

It is an object of the technology disclosed in the present description to provide a surgical instrument capable of switching states of use of treatment portions and a medical manipulator system capable of detachably mounting one or more surgical instruments that can switch states of use of treatment portions are used.

### Solution to Problem

According to a first aspect of the technology disclosed in the present description, there is provided a surgical instrument including a treatment unit having a first treatment portion on an end portion thereof and a second treatment portion on another end portion thereof; and a holder that supports the treatment unit so as to allow states of use of the first treatment portion and the second treatment portion to be switched. The holder supports the holder so as to be rotatable around a switching shaft for switching the states of use of the first treatment portion and the second treatment portion, and supports the treatment unit such that, while one of the first treatment portion and the second treatment portion is in a usable state, the other is in a standby state. Each of the first treatment portion and the second treatment portion may include, for example, any one of forceps, an aeroperitoneum tube, an energy treatment instrument, tweezers, a retractor.

The holder includes a storage portion that stores one of the first treatment portion and the second treatment portion that is in the standby state. The holder supports the treatment unit in a manner allowing the treatment unit to be rotatable around a switching shaft, and the treatment unit is rotated around the switching shaft to switch the first treatment portion and the second treatment portion alternately between the usable state and the standby state. The surgical instrument according to the first aspect further includes a switching mechanism that rotates the treatment unit around the switching shaft.

At least one of the first treatment portion and the second treatment portion includes an openable and closable end effector, and the storage portion stores the openable and closable end effector therein in such a manner that the openable and closable end effector remains closed when the openable and closable end effector is in the standby state. The storage portion has a cross-sectional U shape having an opening along a direction around the switching shaft, and the openable and closable end effector is openable and closable around an opening and closing shaft perpendicular to the switching shaft.

The surgical instrument according to the first aspect may include an opening and closing mechanism that opens and closes the openable and closable end effector. The opening and closing mechanism closes the openable and closable end effector when the forceps transition to the standby state.

Further, according to a second aspect of the technology disclosed in the present description, there is provided a medical manipulator system including a surgical instrument that includes a treatment unit having a first treatment portion on an end portion thereof and a second treatment portion on another end portion thereof, and a holder that supports the treatment unit so as to allow states of use of the first treatment portion and the second treatment portion to be switched, and an arm with the surgical instrument attached thereto, the arm including at least one joint.

Incidentally, the term “system” referred to herein means a logical collection of a plurality of apparatuses (or functional modules for performing particular functions), and it does not matter whether the apparatuses or the functional modules are disposed in a single housing or not.

### Advantageous Effects of Invention

According to the technology disclosed in the present description, there are provided a surgical instrument having a plurality of methods of use and reducing the number of times that treatment portions are detachably replaced and a medical manipulator system incorporating a surgical instrument having a plurality of methods of use.

The advantageous effects set forth in the present description are given by way of illustrative example only and are not restrictive of the advantageous effects of the present invention. Further, the present invention may further produce additional advantageous effects in addition to the abovementioned advantageous effects.

Other objects, features, and advantages of the technology disclosed in the present description will become evident from a more detailed description based on the embodiments to be described later and the accompanying drawings.

### BRIEF DESCRIPTION OF DRAWINGS

FIG. 1 is a view schematically illustrating a structural example of a surgical instrument 100 (first embodiment).

FIG. 2 is a view schematically illustrating the structural example of the surgical instrument 100 (first embodiment).

FIG. 3 is a view illustrating the manner in which states of a first treatment portion 111 and a second treatment portion 112 are switched.

FIG. 4 is a view illustrating the manner in which the states of the first treatment portion 111 and the second treatment portion 112 are switched.

FIG. 5 is a view illustrating the manner in which the states of the first treatment portion 111 and the second treatment portion 112 are switched.

FIG. 6 is a view illustrating the manner in which the states of the first treatment portion 111 and the second treatment portion 112 are switched.

FIG. 7 is a view schematically illustrating a structural example of a surgical instrument 700 (second embodiment).

FIG. 8 is a view schematically illustrating the structural example of the surgical instrument 700 (second embodiment).

FIG. 9 is a view illustrating the manner in which states of first forceps 711 and second forceps 712 are switched.

FIG. 10 is a view illustrating the manner in which the states of the first forceps 711 and the second forceps 712 are switched.

FIG. 11 is a view illustrating the manner in which the states of the first forceps 711 and the second forceps 712 are switched.

FIG. 12 is a view illustrating the manner in which the states of the first forceps 711 and the second forceps 712 are switched.

FIG. 13 is a view schematically illustrating a structural example of a switching mechanism 750.

FIG. 14 is a view schematically illustrating the structural example of the switching mechanism 750.

FIG. 15 is a view schematically illustrating a structural example of the first forceps 711.

FIG. 16 is a view illustrating the manner in which the forceps 711 illustrated in FIG. 15 are closed.

FIG. 17 is a view schematically illustrating a structural example of an opening and closing mechanism 760.

FIG. 18 is a view schematically illustrating an operational example of the opening and closing mechanism 760.

FIG. 19 is a view schematically illustrating a structural example of an opening and closing mechanism 760 that has a function of closing blades.

FIG. 20 is a view schematically illustrating an operational example of the opening and closing mechanism 760 illustrated in FIG. 19.

FIG. 21 is a view schematically illustrating another structural example of an opening and closing mechanism 760 that has a function of closing blades.

FIG. 22 is a view illustrating the manner in which the opening and closing mechanism 760 illustrated in FIG. 21 operates.

FIG. 23 is a view illustrating the manner in which the opening and closing mechanism 760 illustrated in FIG. 21 opens and closes forceps.

FIG. 24 is a view illustrating the manner in which the opening and closing mechanism 760 illustrated in FIG. 21 opens and closes the forceps.

FIG. 25 is a view illustrating the manner in which the opening and closing mechanism 760 illustrated in FIG. 21 closes the forceps that transition to a standby state.

FIG. 26 is a view illustrating the manner in which the opening and closing mechanism 760 illustrated in FIG. 21 closes the forceps that transition to the standby state.

FIG. 27 is a view illustrating the manner in which the opening and closing mechanism 760 illustrated in FIG. 21 closes the forceps that transition to the standby state.

FIG. 28 is a view illustrating the manner in which the opening and closing mechanism 760 illustrated in FIG. 21 closes the forceps that transition to the standby state.

FIG. 29 is a view schematically illustrating a structural example of a surgical instrument 2900 (third embodiment).

FIG. 30 is a view illustrating the manner in which first forceps 2911 are opened and closed.

FIG. 31 is a view illustrating the manner in which the first forceps 2911 are opened and closed.

FIG. 32 is a view illustrating the manner in which the first forceps 2911 are opened and closed.

FIG. 33 is a view illustrating an internal structure of the first forceps 2911.

FIG. 34 is a view illustrating the internal structure of the first forceps 2911.

FIG. 35 is a view illustrating the internal structure of the first forceps 2911.

FIG. 36 is a view illustrating a structure and operation of an opening and closing mechanism applied to the surgical instrument 2900.

FIG. 37 is a view illustrating the structure and operation of the opening and closing mechanism applied to the surgical instrument 2900.

FIG. 38 is a view illustrating the structure and operation of the opening and closing mechanism applied to the surgical instrument 2900.

FIG. 39 is a view illustrating the structure and operation of the opening and closing mechanism applied to the surgical instrument 2900.

FIG. 40 is a view illustrating the structure and operation of the opening and closing mechanism applied to the surgical instrument 2900.

FIG. 41 is a view illustrating an operation to close blades when the first forceps 711 transition from a usable state to a standby state.

FIG. 42 is a view illustrating at an enlarged scale a cam slider 3600 and nearby parts in the state illustrated in FIG. 41.

FIG. 43 is a view illustrating an operation to close the blades when the first forceps 711 transition from the usable state to the standby state.

FIG. 44 is a view illustrating at an enlarged scale the manner in which the cam slider 3600 and nearby parts operate in the state illustrated in FIG. 43.

FIG. 45 is a view illustrating an operation to close the blades when the first forceps 711 transition from the usable state to the standby state.

FIG. 46 is a view illustrating at an enlarged scale the manner in which the cam slider 3600 and nearby parts operate in the state illustrated in FIG. 45.

FIG. 47 is a view illustrating an operation to close the blades when the first forceps 711 transition from the usable state to the standby state.

FIG. 48 is a view illustrating at an enlarged scale the manner in which the cam slider 3600 and nearby parts operate in the state illustrated in FIG. 47.

FIG. 49 is a view schematically illustrating a structural example of a multi-joint arm 4900.

FIG. 50 is a view schematically illustrating a functional configuration of a master-slave surgical system 5000.

### DESCRIPTION OF EMBODIMENTS

Embodiments of the technology disclosed in the present description will be described in detail hereinbelow with reference to the drawings.

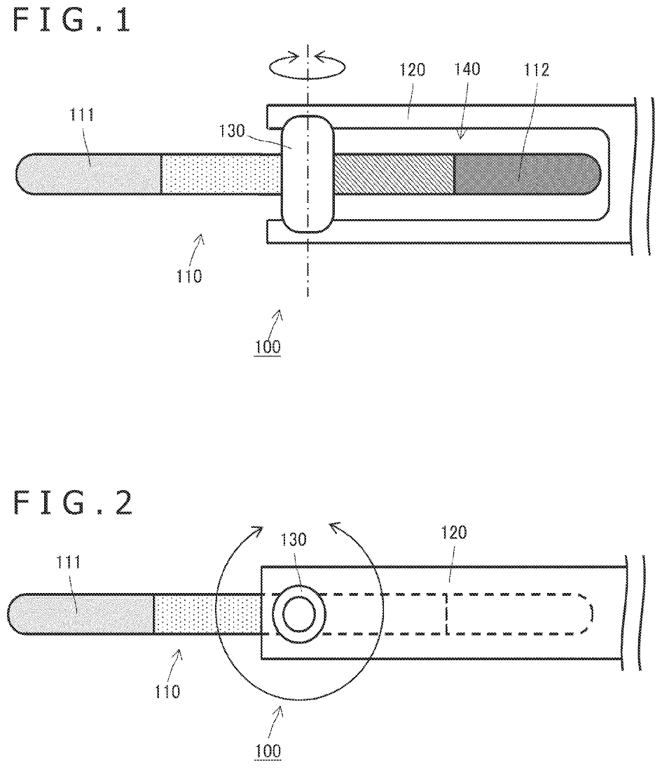

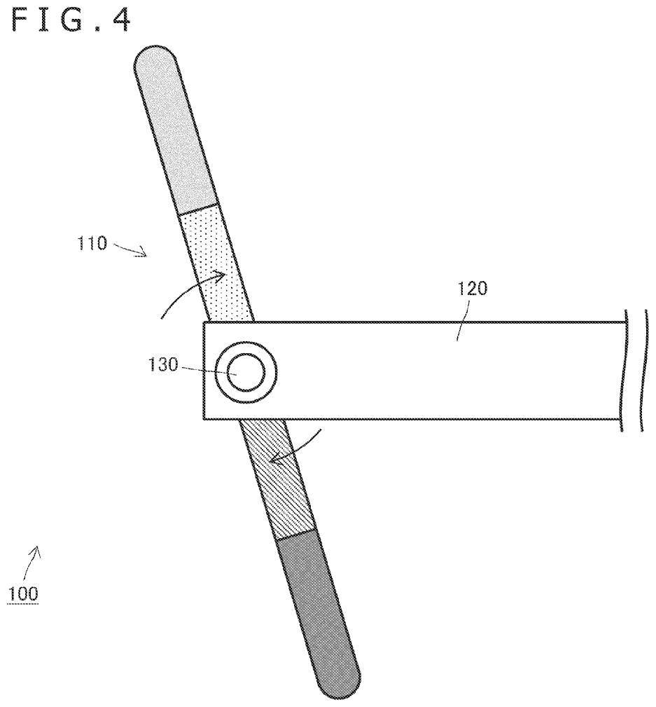

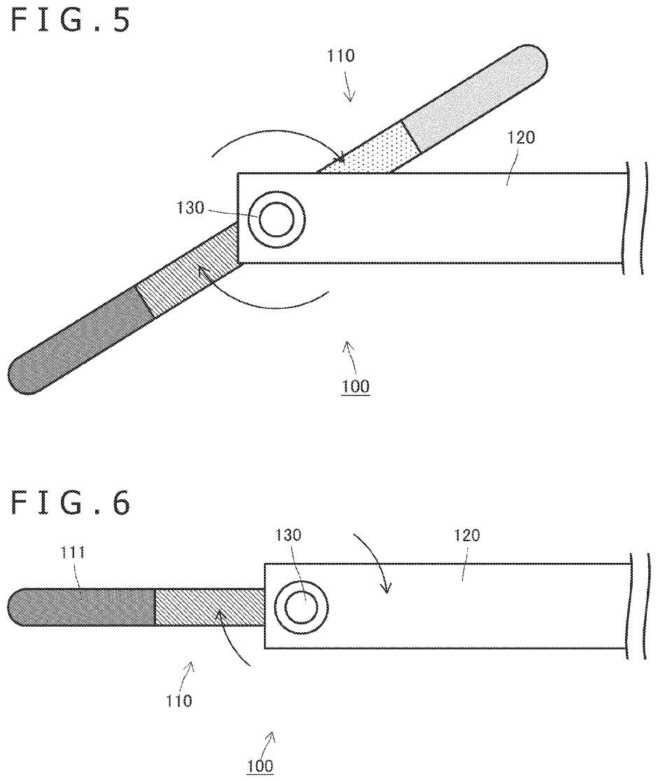

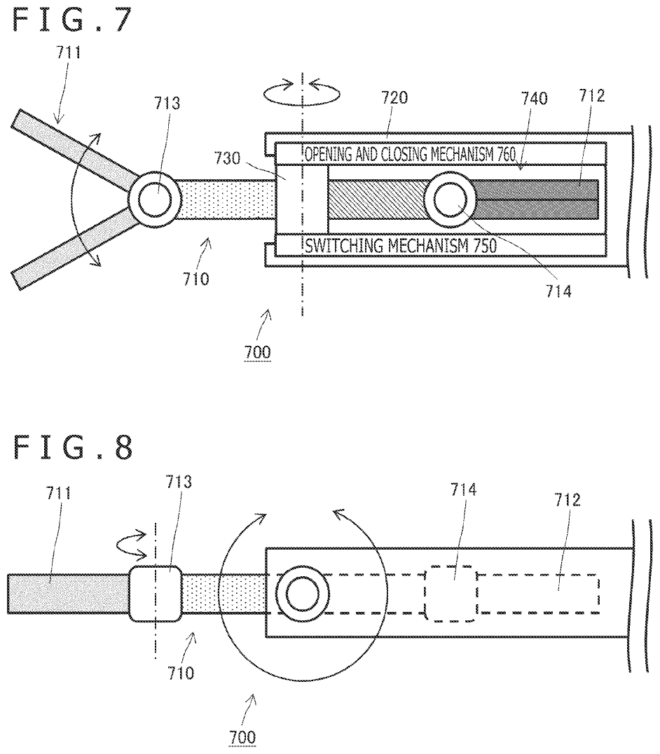

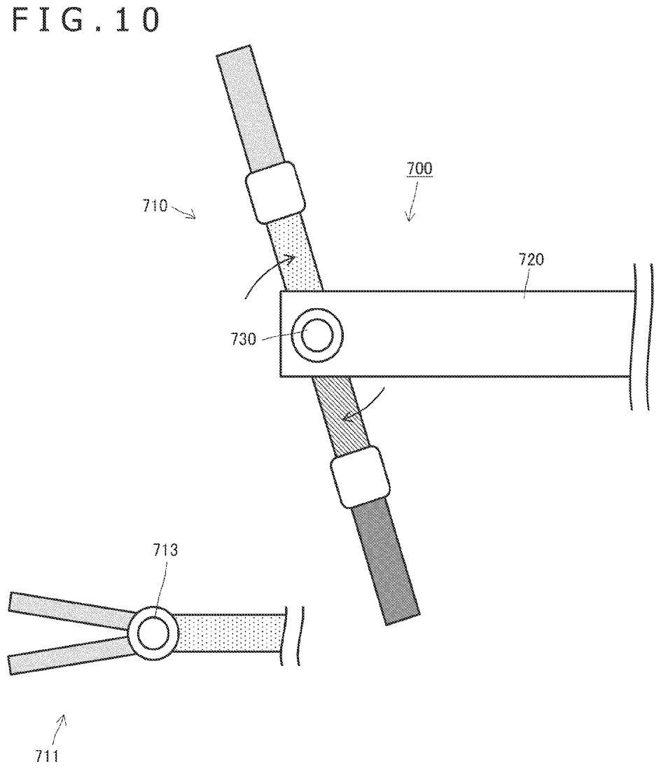

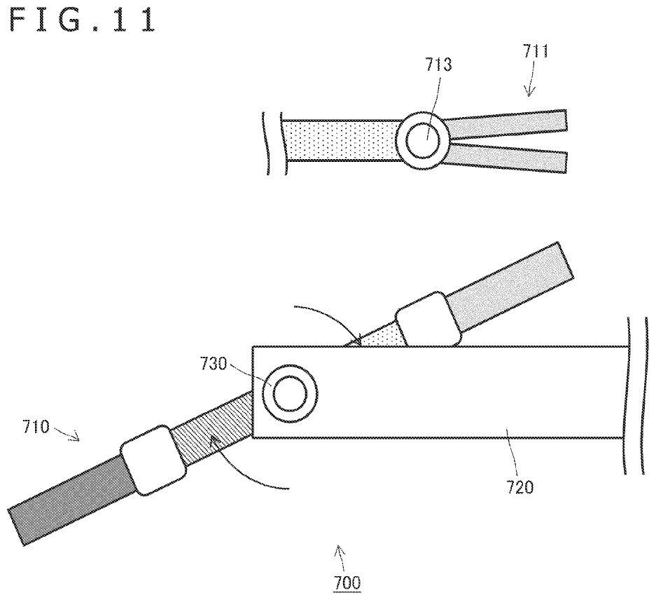

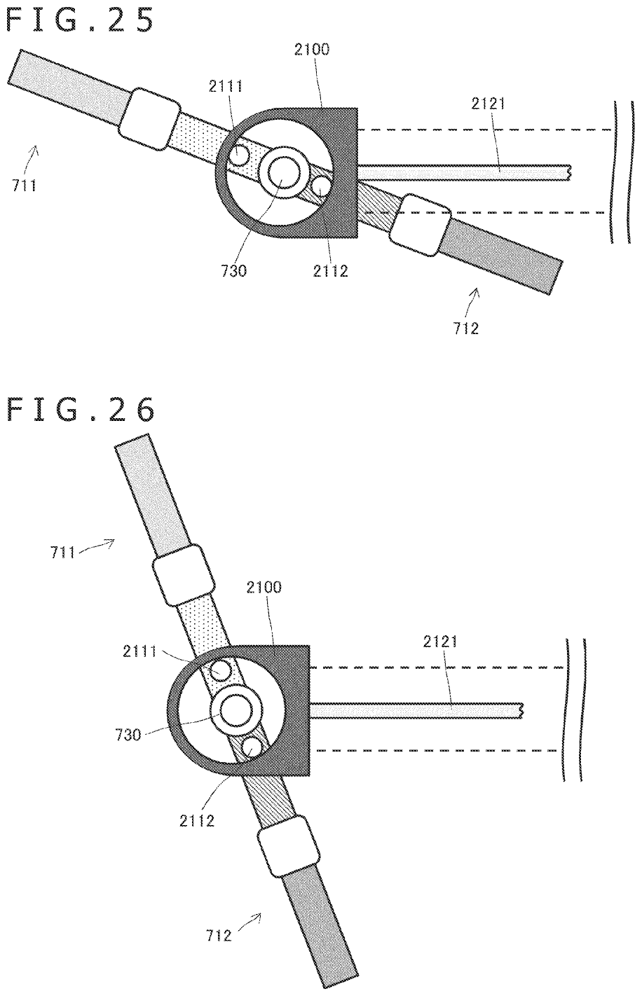

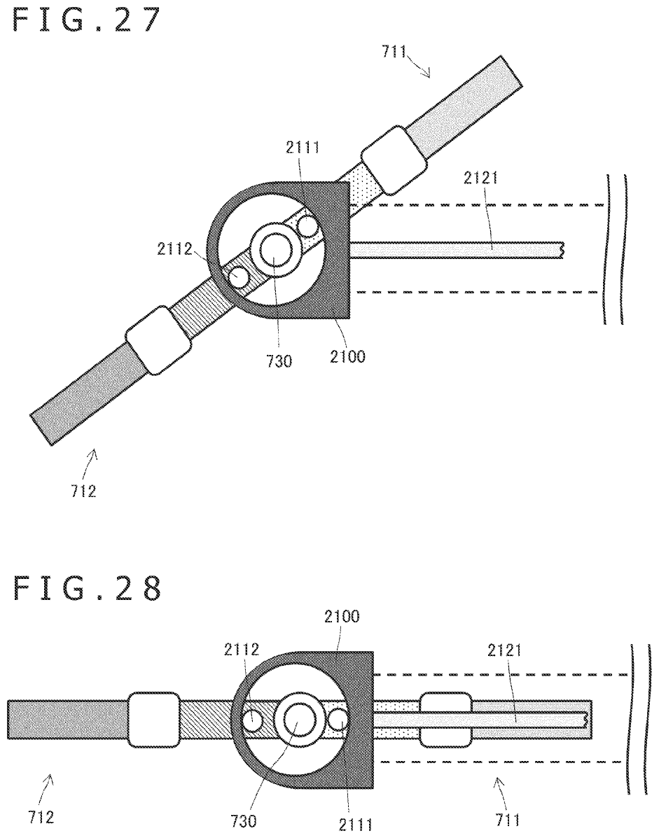

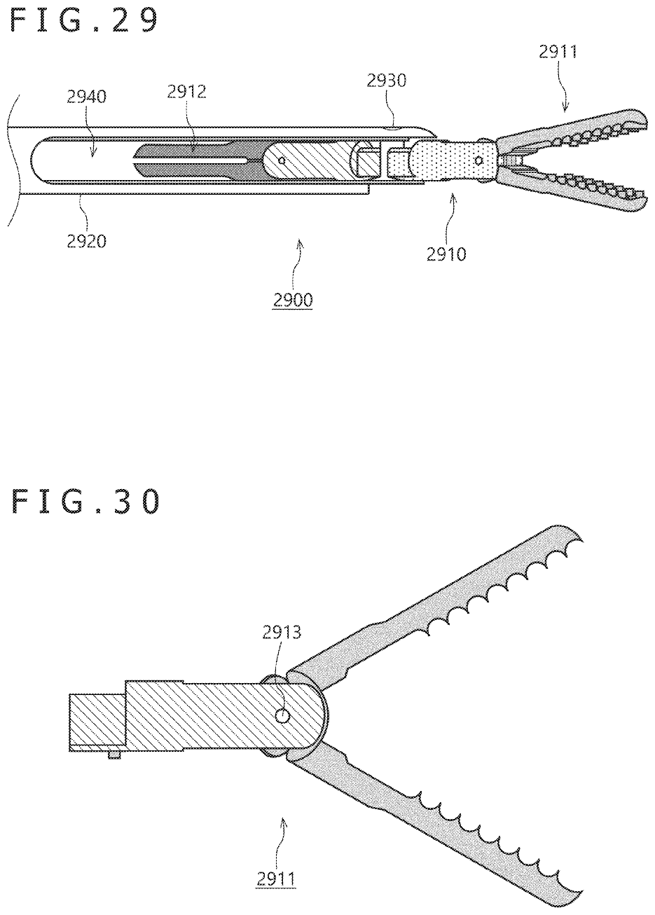

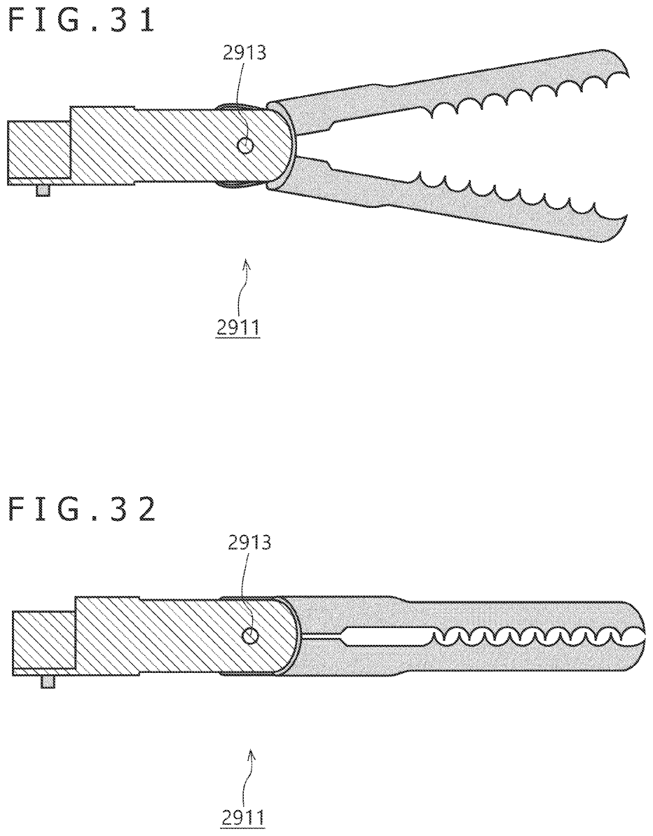

First, a first embodiment of the technology disclosed in the present description will be described with reference to FIGS. 1 through 6. Then, a second embodiment of the technology disclosed in the present description will be described with reference to FIGS. 7 through 28. Further, a third embodiment representing a specific example where the second embodiment is reduced to practice will be described with reference to FIGS. 29 through 48. Still further, a fourth embodiment representing a surgical system that can use surgical instruments illustrated as the first through third embodiments will be described.

### Embodiment 1

FIGS. 1 and 2 schematically illustrate a structural example of a surgical instrument 100 according to the first embodiment. The surgical instrument 100 illustrated in FIGS. 1 and 2 includes a treatment unit 110 and a holder 120. FIG. 1 illustrates the surgical instrument 100 along a plane as viewed sideways that includes the longitudinal directions of a switching shaft 130 (to be described later), and the treatment unit 110, and FIG. 2 illustrates an upper surface of the surgical instrument 100 as viewed along the switching shaft 130.

The treatment unit 110 has a first treatment portion 111 on an end portion thereof and a second treatment portion 112 on another end portion thereof. In FIG. 1, the first treatment portion 111 and the second treatment portion 112 are illustrated as simplified. These treatment portions each include a treatment instrument to be inserted and used in a body cavity of a patient in laparoscopic surgery, such as forceps, an aeroperitoneum tube, an energy treatment instrument, tweezers, a retractor, or the like that is an openable and closable end effector. The energy treatment instrument referred to here is a treatment instrument for incising or peeling a tissue or sealing a blood vessel with a high-frequency current or ultrasonic vibrations.

The treatment unit 110 may have treatment instruments of different kinds, treatment instruments of one kind and different types, or treatment instruments of one kind and one type as the first treatment portion 111 and the second treatment portion 112 on both end portions. At any rate, the treatment unit 110 can be used in two ways with the first treatment portion 111 and the second treatment portion 112.

In order for the user to visually confirm with ease the kind or kinds of the treatment instruments on both end portions of the treatment unit 110, different colors, numbers, letters, prints, or surface states may be defined for their kind, kinds, or types. According to the example illustrated in FIGS. 1 and 2, the first treatment portion 111 is painted out in pale gray and its shank fully stippled, whereas the second treatment portion 112 is painted out in dark gray and its shank fully hatched.

The holder 120 supports the first treatment portion 111 and the second treatment portion 112 so as to allow their states of use to be switched. The states of use referred to here include a usable state and a standby state. The holder 120 supports the treatment unit 110 such that, while one of the first treatment portion 111 and the second treatment portion 112 is in the usable state, the other is in the standby state where it is in storage. Specifically, the holder 120 has a storage portion 140 in the form of an opening having a U-shaped cross section, and the switching shaft 130 is positioned in the vicinity of the distal end of the holder 120 and has the treatment unit 110 rotatably supported thereon. Further, the storage portion 140 has a size and shape that allows either one of the first treatment portion 111 and the second treatment portion 112 to be inserted into the storage portion 140 along a rotational direction around the switching shaft 130. Thus, depending on the angle through which the treatment unit 110 turns around the switching shaft 130, one of the first treatment portion 111 and the second treatment portion 112 emerges out of the storage portion 140 into the usable state, and the other enters the storage portion 140 where it is in the standby state and cannot be used.

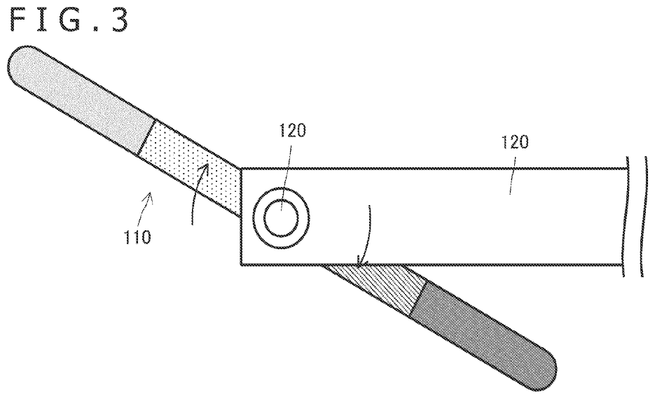

According to the example illustrated in FIGS. 1 and 2, the first treatment portion 111 is in the usable state, whereas the second treatment portion 112 is in the standby state where it is stored in the storage portion 140. Further, the states of the first treatment portion 111 and the second treatment portion 112 are switched around when the treatment unit 110 turns around the switching shaft 130. FIGS. 3 through 6 illustrate stepwise the manner in which, when the treatment unit 110 turns 180 degrees around the switching shaft 130 clockwise in the plane of the sheets of FIGS. 3 through 6, the second treatment portion 112 switches from the standby state to the usable state and at the same time the first treatment portion 111 switches from the usable state to the standby state. As can be recognized from FIGS. 2 through 6, when the treatment unit 110 turns around the switching shaft 130 through 180 degrees, the states of the first treatment portion 111 and the second treatment portion 112 are switched around.

Incidentally, the surgical instrument should preferably include for its usage a mechanism such as a latch (not illustrated) for holding the treatment unit 110 against rotation around the switching shaft 130 at an angular position where one of the first treatment portion 111 and the second treatment portion 112 is in the usable state whereas the other is in the standby state (or when either the first treatment portion 111 or the second treatment portion 112 is in the usable state), as illustrated in FIGS. 2 and 6.

In addition, in each of FIGS. 1 through 6, the holder 120 has its proximal portion omitted from illustration beyond the left end because of the limited space available. It is assumed that the proximal end portion of the holder 120 is of a shape and structure on which the slave arm of a surgical master-slave system, for example, can be mounted. Alternatively, the proximal end portion of the holder 120 may be of a shape and structure that can be attached to manual forceps that are manually operable.

When the states of the first treatment portion 111 and the second treatment portion 112 are successfully switched, the successful switching may be indicated to the user (the surgeon or the like). For example, an encoder for detecting the angular position of the treatment unit 110 around the switching shaft 130, another sensor, or the like may be used to detect when either the first treatment portion 111 or the second treatment portion 112 has been set in the usable state. Then, the detected result is output to the master side of the surgical master-slave system, and the master device indicates to the user that the switching of the treatment portion has been completed, by way of illumination (e.g., LED (Light Emitting Diode) blinking), sound (e.g., beeping), haptic technology, monitor screen display, or the like.

The surgical instrument 100 according to the first embodiment is used in laparoscopic surgery, for example, by being inserted through a trocar into a body cavity of the patient. For example, the surgeon may confirm a space in the body cavity large enough to turn the treatment unit 110 with safety therein and may then switch the treatment portions in the body cavity without removing the surgical instrument 100 from the body cavity. Alternatively, the surgical instrument 100 may incorporate a distance sensor (a TOF (Time Of Flight) distance sensor, a laser distance sensor, an IR (Infrared) distance sensor, or the like), and the surgeon may measure the distance up to surrounding tissues with the distance sensor, confirm a space in the body cavity large enough to turn the treatment unit 110 with safety therein, and then switch the treatment portions in the body cavity.

The surgical instrument 100 according to the first embodiment can be used in two ways with the first treatment portion 111 and the second treatment portion 112, respectively, by rotating the treatment unit 110 through 180 degrees (or stated otherwise, without removing the surgical instrument 100 from the body cavity and switching the treatment instruments as in the conventional art). Thus, a plurality of treatment instruments can be used on the same surgical instrument in a space saving environment. Further, the treatment instruments can be switched around in a short period of time by simply turning the treatment unit 110. Consequently, the time required for the surgical operation is reduced by reducing the time required for switching the surgical instrument, resulting in a commensurate reduction in the burden on the patient.

In a case where a surgical operation is carried out using surgical instruments 100 according to the first embodiment mounted on a surgical robot, the number of surgical instruments to be installed on the surgical robot can be reduced, or, in a case where the same number of surgical instruments as in the past are installed on the surgical robot, more treatment instruments can simultaneously be used. Further, if the surgical instrument 100 is sterilized or cleaned, then the two kinds of treatment instruments are simultaneously sterilized or cleaned. In addition, as the number of times that surgical instruments on the surgical robot are replaced is reduced, the time required for changing steps of the surgical operation is shortened, reducing the trouble that the surgeon has to take.

### Embodiment 2

A surgical instrument capable of switching states in which two kinds of openable and closable end effectors are used according to a second embodiment will be described below. Since a pair of forceps, for example, have an openable and closable structure, the feature of the surgical instrument according to the second embodiment resides in that not only is it able to changes states in which two pairs of forceps are used, but it has also a function of opening and closing the forceps in a usable state and a function of controlling opened and closed states of the forceps at the time their states of use are switched. The end effectors may be jaws, cutting blades, staplers, or the like for generating gripping forces, for example. The surgical instrument may alternatively be one kind of an openable and closable end effector combined with an end effector that is not of the openable and closable type (e.g., an energy treatment instrument), instead of two kinds of openable and closable end effectors.

FIGS. 7 and 8 schematically illustrate a structural example of a surgical instrument 700 according to the second embodiment. The surgical instrument 700 illustrated in FIGS. 7 and 8 includes a treatment unit 710 and a holder 720. FIG. 7 illustrates the surgical instrument 700 along a plane as viewed sideways that includes the longitudinal directions of a switching shaft 730 (to be described later) and the treatment unit 710, and FIG. 8 illustrates an upper surface of the surgical instrument 700 as viewed along the switching shaft 730.

The treatment unit 710 includes first forceps 711 on an end portion thereof and second forceps 712 on another end portion thereof. As well known in the art, the forceps are of a structure (a pair of jaws) that can be opened and closed and that includes a pair of blades (jaws) each pivotally supported for rotation about an opening and closing shaft in the vicinity of its proximal end. In FIG. 7, the blades of the first forceps 711 and the second forceps 712 are illustrated as simplified in shape for simplification of the drawing, though they may actually have different shapes depending on objects to be gripped or applications in which they are used.

In order for the user to visually confirm with ease the kind or kinds of the forceps on both end portions of the treatment unit 710, different colors, numbers, letters, prints, or surface states may be defined for their kind, kinds, or types. According to the example illustrated in FIGS. 7 and 8, the first forceps 711 are painted out in pale gray and their shank fully stippled, whereas the second forceps 712 are painted out in dark gray and their shank fully hatched.

The holder 720 supports the first forceps 711 and the second forceps 712 so as to allow their states of use to be switched. The states of use referred to here include a usable state and a standby state. The holder 720 supports the treatment unit 710 such that, while one pair of the first forceps 711 and the second forceps 712 is in the usable state, the other pair is in the standby state where it is in storage. Specifically, the holder 720 has a storage portion 740 in the form of an opening having a U-shaped cross section, and the switching shaft 730 is positioned in the vicinity of the distal end of the holder 720 and has the treatment unit 710 rotatably supported thereon. Further, the storage portion 740 has such a size and shape that allows any pair of the first forceps 711 and the second forceps 712 to be inserted into the storage portion 740 along a rotational direction around the switching shaft 730. Thus, depending on the angle through which the treatment unit 710 turns around the switching shaft 730, one pair of the first forceps 711 and the second forceps 712 emerges out of the storage portion 740 into the usable state, and the other pair enters the storage portion 740 where it is in the standby state and cannot be used.

According to the example illustrated in FIGS. 7 and 8, the first forceps 711 are in the usable state, whereas the second forceps 712 are in the standby state where they are stored in the storage portion 740. In addition, the states of the first forceps 711 and the second forceps 712 can be switched when the treatment unit 710 turns around the switching shaft 730. The surgical instrument 700 includes a switching mechanism 750 for rotating the treatment unit 710 around the switching shaft 730 to switch the states of the first forceps 711 and the second forceps 712. However, details of the switching mechanism 750 will be described later.

The first forceps 711 are of a structure including a pair of blades each pivotally supported for rotation around an opening and closing shaft 713 in the vicinity of its proximal end, so that the blades can be opened and closed. Similarly, the second forceps 712 are of a structure including a pair of blades each pivotally supported for rotation around an opening and closing shaft 714 in the vicinity of its proximal end, so that the blades can be opened and closed.

Both the opening and closing shafts 713 and 714 of the first forceps 711 and the second forceps 712 are perpendicular to a rotational axis of the switching shaft 730. Thus, both the first forceps 711 and the second forceps 712 have their blades openable and closable in directions parallel to the rotational axis of the switching shaft 730. Stated otherwise, the first forceps 711 and the second forceps 712 are openable and closable in directions perpendicular to the path followed by the treatment unit 710 when the treatment unit 710 turns around the switching shaft 730.

The storage portion 740 is of a U shape having an opening along the path followed by a distal end of the treatment unit 710. When the blades of the first forceps 711 and the second forceps 712 are closed upon their transition from the usable state to the standby state, the first forceps 711 and the second forceps 712 can be placed into the U shape of the storage portion 740. Further, once stored in the storage portion 740, as the blades are kept within the U shape, they cannot be opened and closed. According to the example illustrated in FIGS. 7 and 8, the first forceps 711 that are in the usable state stay outside of the storage portion 740 and have their blades openable and closable. On the other hand, since the second forceps 712 that are in the standby state are stored in the storage portion 740, their blades remain closed and cannot be opened.

The surgical instrument 700 includes an opening and closing mechanism 760 for opening and closing those of the first forceps 711 and the second forceps 712 that are in the usable state. The opening and closing mechanism 760 also makes a closing motion with the blades when the first forceps 711 and the second forceps 712 transition from the usable state to the standby state. This is because the blades would impinge upon the U-shaped wall of the storage portion 740 if they remain opened. Details of the opening and closing mechanism 760 will be described later.

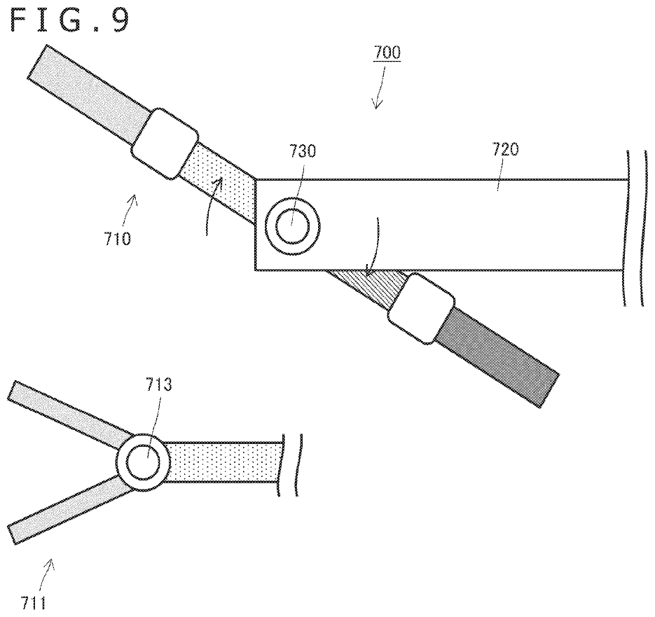

FIGS. 9 through 12 illustrate stepwise the manner in which the treatment unit 710 turns around the switching shaft 730 clockwise in the plane of the sheets of FIGS. 9 through 12. As can be recognized from FIGS. 8 through 12, when the treatment unit 710 turns 180 degrees around the switching shaft 730, the second forceps 712 switch from the standby state to the usable state while at the same time the first forceps 711 switch from the usable state to the standby state. In addition, each of FIGS. 9 through 12 also illustrates the first forceps 711 as viewed sideways, i.e., along the opening and closing shaft 713. It should be fully understood that the blades are gradually closed in the course of a transition of the first forceps 711 from the usable state to the standby state.

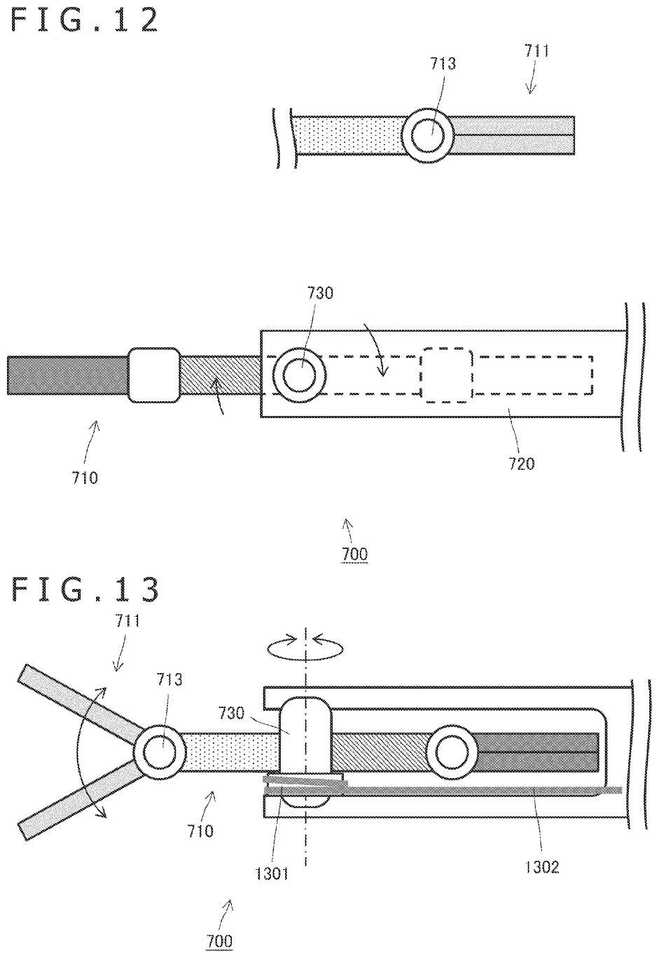

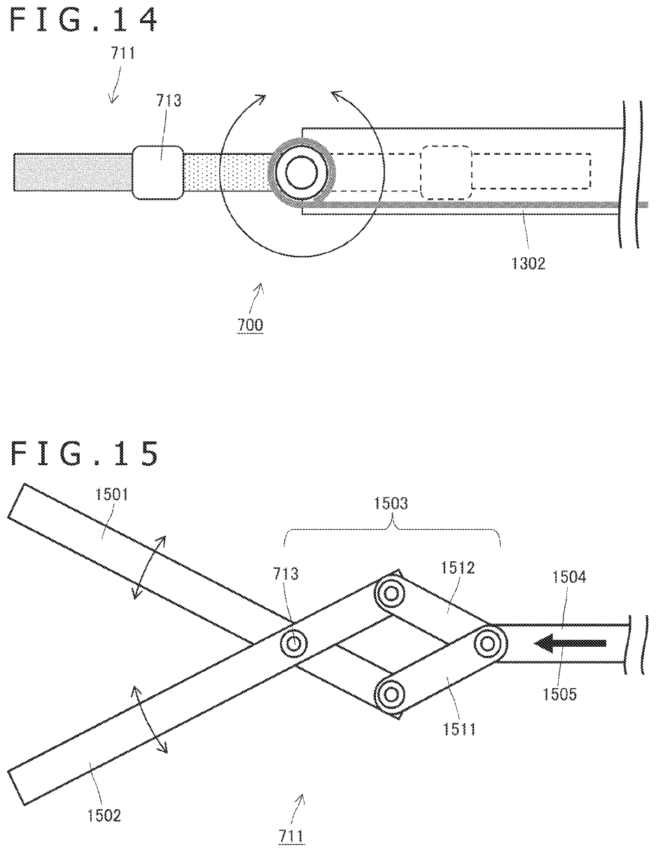

FIGS. 13 and 14 illustrate a structural example of a switching mechanism 750 as applied to the surgical instrument 700 described above. According to the example illustrated in FIGS. 13 and 14, the switching mechanism 750 includes a pulley 1301 rotatable in unison with the treatment unit 710 around the switching shaft 730 and a wire 1302 wound in a turn or a plurality of turns around the outer circumferential surface of the pulley 1301. When the wire 1302 is pulled from the proximal end side of the surgical instrument 700, the treatment unit 710 turns around the switching shaft 730. Further, when the treatment unit 710 turns 180 degrees around the switching shaft 730, the states of the first forceps 711 and the second forceps 712 are switched around.

FIG. 15 schematically illustrates a structural example of the first forceps 711. An opening and closing mechanism for the first forceps 711 will be described below with reference to FIG. 15. Note that, though details are omitted from illustration with respect to the second forceps 712, an opening and closing mechanism for the second forceps 712 may have a structure similar to the structural example illustrated in FIG. 15 except for differences with the shapes of the blade edges of the blades and the like.

The first forceps 711 include a pair of blades 1501 and 1502. The blades 1501 and 1502 cross each other at an opening and closing shaft 713 and can turn around the opening and closing shaft 713. Links 1511 and 1512 are added to respective portions of the blades 1501 and 1502 that are closer to the proximal end side of the first forceps 711 than the opening and closing shaft 713, constituting a parallel crank 1503. Further, a linear-motion portion 1504 that is slidable back and forth in the longitudinal directions of the treatment unit 710 is coupled to a joint portion by which the links 1511 and 1512 are joined to each other.

Normally, the linear-motion portion 1504 is pushed in the direction of a distal end side (a far end side) indicated by the arrow denoted by the reference numeral 1505 under the returning force of a spring (not illustrated). Thus, the parallel crank 1503 has its lozenge shape opened, applying, in ganged relation thereto, a force tending to open the blades 1501 and 1502 around the opening and closing shaft 713. Therefore, the first forceps 711 are open as illustrated in FIG. 15 in the absence of a force applied thereto.

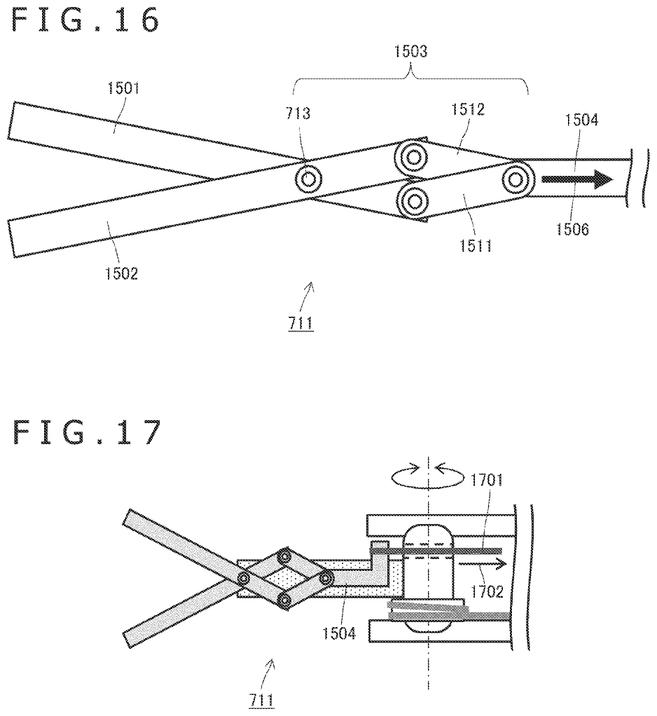

When the linear-motion portion 1504 near the proximal end side of the parallel crank 1503 is pulled in the direction of the proximal end side (a near end side) indicated by the arrow denoted by the reference numeral 1506, the lozenge shape of the parallel crank 1503 is folded, applying, in ganged relation thereto, a force tending to close the blades 1501 and 1502 around the opening and closing shaft 713. Therefore, the first forceps 711 are closed as illustrated in FIG. 16.

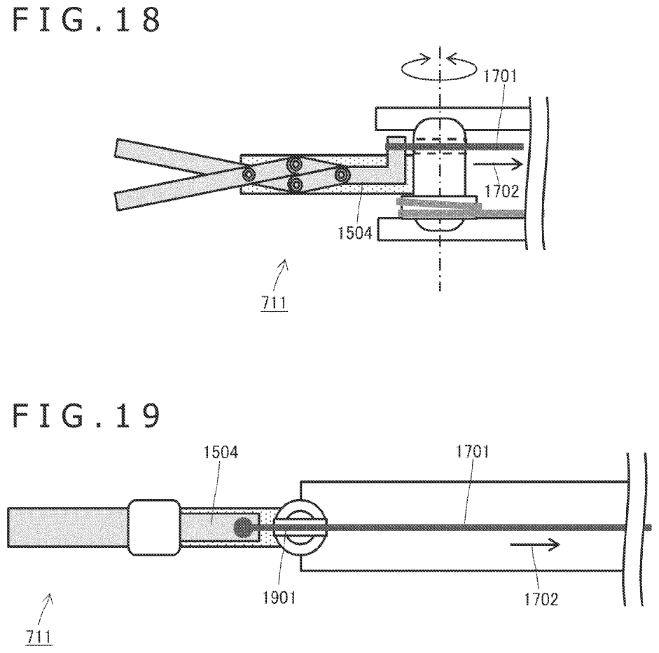

FIGS. 17 and 18 illustrate a structural example of the opening and closing mechanism 760 that incorporates the first forceps 711 having the structure illustrated in FIGS. 15 and 16, and also illustrate the manner in which the first forceps 711 are opened and closed. According to the example illustrated in FIGS. 17 and 18, the opening and closing mechanism 760 includes a wire 1701 attached to the linear-motion portion 1504. The linear-motion portion 1504 can be pulled in the direction of the proximal end side (the near end side) by the wire 1701.

Normally, the linear-motion portion 1504 is pushed in the direction of the distal end side (the far end side) indicated by the arrow denoted by the reference numeral 1505 under the returning force of a spring (not illustrated), opening the first forceps 711, as illustrated in FIG. 17. Here, when the linear-motion portion 1504 is pulled in the direction of the proximal end side (the far end side) through the wire 1701, the lozenge shape of the parallel crank 1503 is folded, applying, in ganged relation thereto, a force tending to close the blades 1501 and 1502 around the opening and closing shaft 713. Therefore, the first forceps 711 are closed as illustrated in FIG. 18.

In addition, the opening and closing mechanism 760 should desirably have a function of making a closing motion with the blades when the first forceps 711 transition from the usable state to the standby state, i.e., while the treatment unit 710 turns 180 degrees around the switching shaft 730 (as described above). This is because the blades would impinge upon the U-shaped wall of the storage portion 740 if they remain opened.

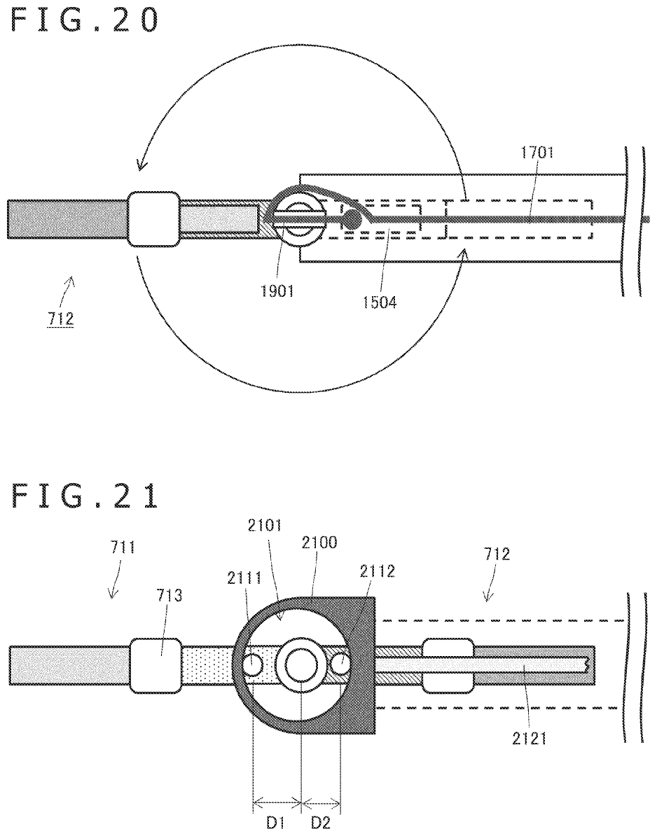

FIGS. 19 and 20 illustrate a structural example of the opening and closing mechanism 760 that has a function of closing the blades while the treatment unit 710 turns 180 degrees around the switching shaft 730. As illustrated in FIG. 19, the switching shaft 730 has an insertion hole 1901 defined therethrough. In addition, the wire 1701 attached to the linear-motion portion 1504 of the first forceps 711 extends through the insertion hole 1901 in the switching shaft 730. Thus, when the first forceps 711 turn 180 degrees around the switching shaft 730 to transition into the standby state, the wire 1701 is wound around the outer circumferential surface of the switching shaft 730, as illustrated in FIG. 20. As a result, the wire 1701 pulls the linear-motion portion 1504, closing the blades of the first forceps 711.

In a case where the pulley of the switching shaft 730 has a diameter of 5 millimeters, for example, when the treatment unit 710 turns 180 degrees, the wire 1701 is pulled a distance of π×r=π×2.5≈7.8 millimeters. The distance that the wire 1701 is pulled upon a state transition can be adjusted by the size and shape of the pulley.

FIG. 21 illustrates another structural example of the opening and closing mechanism 760. According to the illustrated example, the opening and closing mechanism 760 is constructed as a cam slider 2100 having an opening 2101 having an inner circumferential surface as a cam surface, the cam slider 2100 being slidable in the longitudinal directions of the treatment unit 710. Note that the first forceps 711 having the structure illustrated in FIGS. 15 and 16 are used in combination with the opening and closing mechanism 760. The opening and closing mechanism 760 constructed as the cam slider 2100 illustrated in FIG. 21 also has a function of closing the blades while the treatment unit 710 turns 180 degrees around the switching shaft 730.

The opening 2101 in the cam slider 2100 accommodates therein a protrusion 2111 disposed on an end of the linear-motion portion 1504 of the first forceps 711. In addition, a protrusion 2112 disposed on an end of a similar linear-motion portion of the second forceps 712 is also accommodated in the opening 2101. Further, a wire 2121 is attached to an edge of the cam slider 2110 closer to the proximal end side.

The opening 2101 in the cam slider 2100 has a cam profile whose center is disposed eccentrically closer to the forceps in the standby state (or to the near end side) than the center of rotation of the switching shaft 730. In other words, the opening 2101 has a cam profile such that it has a longest radius from the switching shaft 730 in the direction of the distal end side (the far end side) and a shortest radius from the switching shaft 730 in the direction of the proximal end side (the near end side).

As illustrated in FIG. 21, when the first forceps 711 are in the usable state and the second forceps 712 are in the standby state, the distance D2 from the protrusion 2112 to the center of rotation of the switching shaft 730 is shorter than the distance D1 from the protrusion 2111 to the center of rotation of the switching shaft 730. Thus, since the linear-motion portion 1504 of the first forceps 711 that are in the usable state is not pulled toward the proximal end side, the first forceps 711 can be opened and closed. On the other hand, the linear-motion portion of the second forceps 712 that are in the standby state is pulled in the direction opposite the blades by the protrusion 2112 held in abutment against the inner wall surface of the opening 2101. As a result, the blades of the second forceps 712 in the standby state can be closed.

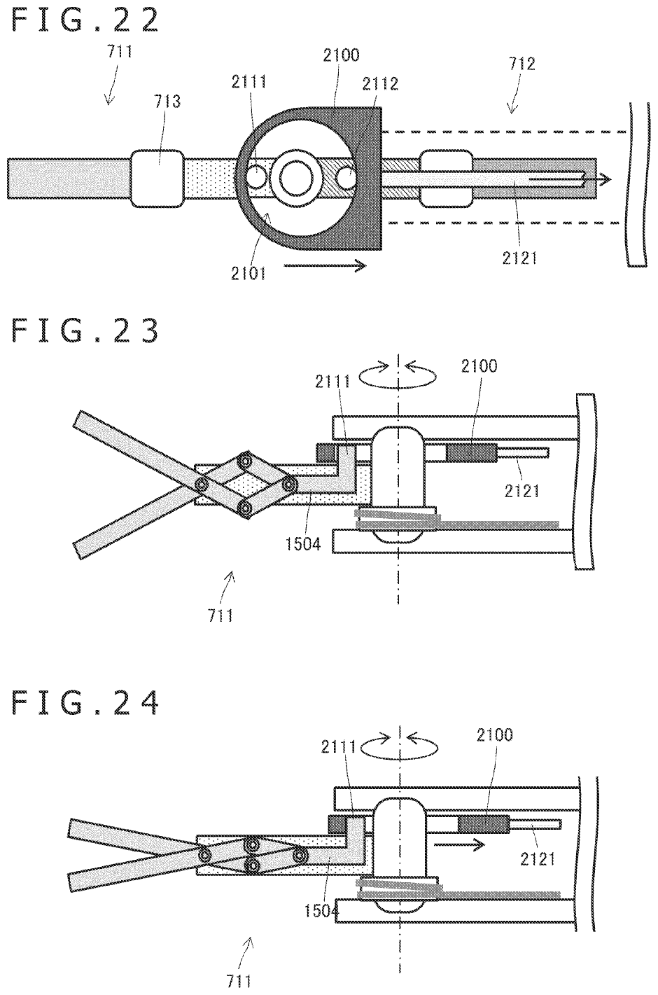

First, opening and closing motions of the forceps that are made by the opening and closing mechanism 760 illustrated in FIG. 21 will be described below with reference to FIGS. 21 through 24. Note that, FIG. 23 illustrates, in side elevation, the treatment unit 710 in a case where the opening and closing mechanism 760 is in the position corresponding to that in FIG. 21, and FIG. 24 illustrates, in side elevation, the treatment unit 710 in a case where the opening and closing mechanism 760 is in the position corresponding to that in FIG. 22. In addition, in each of FIGS. 21 through 24, the first forceps 711 are in the usable state.

Normally, the cam slider 2100 is pushed in the direction of a distal end side (a far end side), under the returning force of a spring (not illustrated). Thus, the parallel crank 1503 has its lozenge shape opened, applying, in ganged relation thereto, a force tending to open the blades 1501 and 1502 around the opening and closing shaft 713. Therefore, the first forceps 711 are open as illustrated in FIGS. 21 and 23 in the absence of a force applied thereto.

Then, when the cam slider 2100 is pulled toward a proximal end side (rightwardly on the sheets of FIGS. 22 and 24) by the wire 2121, the linear-motion portion 1504 is pulled by the protrusion 2111 held in abutment against the inner wall surface of the opening 2101. As a consequence, the lozenge shape of the parallel crank 1503 is folded, applying, in ganged relation thereto, a force tending to close the blades 1501 and 1502 around the opening and closing shaft 713. Therefore, the first forceps 711 are closed as illustrated in FIGS. 22 and 24.

Next, motions made by the opening and closing mechanism 760 illustrated in FIG. 21 to close the blades when the first forceps 711 transition from the usable state to the standby state will be described below. FIGS. 25 through 28 illustrate stepwise the manner in which the treatment unit 710 turns 180 degrees around the switching shaft 730 clockwise in the plane of the sheets of FIGS. 25 through 28. As can be understood from FIGS. 21 and 25 through 28, when the treatment unit 710 turns 180 degrees around the switching shaft 730, the second forceps 712 switch from the standby state to the usable state and at the same time the first forceps 711 switch from the usable state to the standby state. Moreover, inasmuch as the protrusion 2111 abuts against and is pulled by the inner wall surface of the opening 2101 depending on the angle through which the treatment unit 710 turns around the switching shaft 730, the blades of the first forceps 711 are gradually closed as the protrusion 2111 is thus moved. Further, when the treatment unit 710 turns through 180 degrees, the blades of the first forceps 711 are completely closed and stored in the U shape of the storage portion 740, so that they are placed in the standby state. The other second forceps 712 switch into the usable state.

Incidentally, the surgical instrument should preferably include for its usage a mechanism such as a latch (omitted from illustration) for holding the treatment unit 710 against rotation around the switching shaft 730 at an angular position where one pair of the first forceps 711 and the second forceps 712 is in the usable state whereas the other pair is in the standby state (or when either the first forceps 711 or the second forceps 712 are in the usable state).

In addition, in each of FIGS. 7 through 28, the holder 720 has its proximal portion omitted from illustration beyond the left end because of the limited space available. It is assumed that the proximal end portion of the holder 720 is of a shape and structure on which the slave arm of a surgical master-slave system, for example, can be mounted. Alternatively, the proximal end portion of the holder 720 may be of a shape and structure that can be attached to manual forceps that are manually operable.

When the states of the first forceps 711 and the second forceps 712 are successfully switched, the successful switching may be indicated to the user (the surgeon or the like). For example, an encoder for detecting the angular position of the treatment unit 710 around the switching shaft 730, another sensor, or the like may be used to detect when either the first forceps 711 or the second forceps 712 have been set in the usable state. Then, the detected result is output to the master side of the surgical master-slave system, and the master device indicates to the user that the switching of the treatment portion has been completed, by way of illumination (e.g., LED (Light Emitting Diode) blinking), sound (e.g., beeping), haptic technology, monitor screen display, or the like.

The surgical instrument 700 according to the second embodiment is used in laparoscopic surgery, for example, by being inserted through a trocar into a body cavity of the patient. For example, the surgeon may confirm a space in the body cavity large enough to turn the treatment unit 710 with safety therein and may then switch the forceps in the body cavity without removing the surgical instrument 700 from the body cavity. Alternatively, the surgical instrument 700 may incorporate a distance sensor (a TOF distance sensor, a laser distance sensor, an IR distance sensor, or the like), and the surgeon may measure the distance up to surrounding tissues with the distance sensor, confirm a space in the body cavity large enough to turn the treatment unit 710 with safety therein, and then switch the forceps in the body cavity without removing the surgical instrument 700 from the body cavity.

The surgical instrument 700 according to the second embodiment can be used in two ways with the first forceps 711 and the second forceps 712 by turning the treatment unit 710 through 180 degrees (stated otherwise, without removing the surgical instrument 700 from the body cavity and changing the forceps as in the conventional art). Thus, a plurality of forceps can be used on the same surgical instrument in a space saving environment. Further, the time required for the surgical operation is reduced by reducing the time required for switching the surgical instrument, resulting in a commensurate reduction in the burden on the patient.

In a case where a surgical operation is carried out using surgical instruments 700 according to the second embodiment mounted on a surgical robot, the number of surgical instruments to be installed on the surgical robot can be reduced, or, in a case where the same number of surgical instruments as in the past are installed on the surgical robot, more treatment instruments can simultaneously be used. Further, if the surgical instrument 700 is sterilized or cleaned, then the two kinds of treatment instruments are simultaneously sterilized or cleaned. In addition, as the number of times that surgical instruments on the surgical robot are replaced is reduced, the time required to change steps of the surgical operation is shortened, reducing the trouble that the surgeon has to take.

### Embodiment 3

According to a third embodiment, as with the second embodiment, a surgical instrument capable of switching the states of use of two kinds of openable and closable treatment portions will be described below. In the third embodiment, however, specific examples in which components of the surgical instrument are mounted in position will be described below with reference to the drawings. Structural examples of specific components for realizing a function of, not simply switching the states of use of two treatment portions, but opening and closing the treatment portions (forceps), in the usable state and a function of controlling the open and closed states of the treatment portions at the time the states of use are switched will also be described in detail below. Note that the surgical instrument may include one kind of an openable and closable treatment portion combined with a treatment portion that is not of the openable and closable type (e.g., an energy treatment instrument), instead of two kinds of openable and closable treatment portions (the latter surgical instrument may have a function of opening and closing the treatment portions and a function of controlling the open and closed states of the treatment portions at the time the states of use are switched around with respect to only the openable and closable treatment portion).

FIG. 29 illustrates a structural example of a surgical instrument 2900 according to the third embodiment. The illustrated surgical instrument 2900 includes a treatment unit 2910 and a holder 2920.

The treatment unit 2910 includes first forceps 2911 on an end portion thereof and second forceps 2912 on another end portion thereof. As well known in the art, the forceps are of a structure (a pair of jaws) that can be opened and closed and that include a pair of blades (jaws) each pivotally supported for rotation about an opening and closing shaft in the vicinity of its proximal end. In FIG. 29, the blades of the first forceps 2911 and the second forceps 2912 have differently shaped gripping surfaces.

In order for the user to visually confirm with ease the kind or kinds of the forceps on both end portions of the treatment unit 2910, different colors, numbers, letters, prints, or surface states may be defined for their kind, kinds, or types. According to the example illustrated in FIG. 29, the first forceps 2911 are painted out in pale gray and their shank fully stippled, whereas the second forceps 2912 are painted out in dark gray and their shank fully hatched.

The holder 2920 supports the first forceps 2911 and the second forceps 2912 so as to allow their states of use to be switched. The states of use referred to here include a usable state and a standby state. The holder 2920 supports the treatment unit 2910 such that, while one pair of the first forceps 2911 and the second forceps 2912 is in the usable state, the other pair is in the standby state where it is in storage. Specifically, the holder 2920 has a storage portion 2940 in the form of an opening having a U-shaped cross section, and a switching shaft 2930 is positioned in the vicinity of the distal end of the holder 2920 and has the treatment unit 2910 rotatably supported thereon. Further, the storage portion 740 has a size and shape that allows either one pair of the first forceps 2911 and the second forceps 2912 to be inserted into the storage portion 2940 along a rotational direction around the switching shaft 2930. Thus, depending on the angle through which the treatment unit 2910 turns around the switching shaft 2930, one pair of the first forceps 2911 and the second forceps 2912 emerges out of the storage portion 2940 into the usable state, and the other pair enters the storage portion 2940 where it is in the standby state and cannot be used.

According to the example illustrated in FIG. 29, the first forceps 2911 are in the usable state, whereas the second forceps 2912 are in the standby state where they are stored in the storage portion 2940. The states of the first forceps 2911 and the second forceps 2912 are switched around when the treatment unit 2910 turns around the switching shaft 2930.

Note that details of a mechanism for turning the treatment unit 2910 around the switching shaft 2930 will be omitted from description hereinbelow. As described in the above second embodiment with reference to FIGS. 13 and 14, for example, such a mechanism may be a rotating mechanism for pulling a wire wound around a pulley integral with the switching shaft 2930.

FIGS. 30 through 32 illustrate stepwise the manner in which the first forceps 2911 are opened and closed. The first forceps 2911 include a pair of blades each pivotally supported for rotation about an opening and closing shaft 2913 in the vicinity of its proximal end. Although not illustrated, the second forceps 2912 also include a pair of blades each pivotally supported for rotation about an opening and closing shaft in the vicinity of its proximal end.

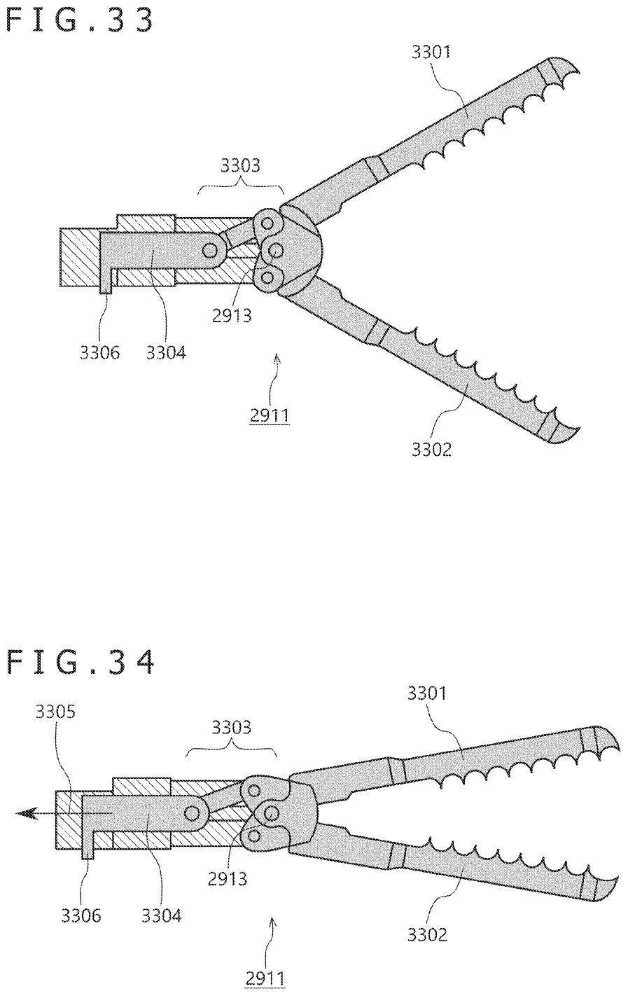

FIGS. 33 through 35 illustrate an internal structure of the first forceps 2911. Though details are omitted from illustration with respect to the second forceps 2912, an opening and closing mechanism for the second forceps 2912 may have a structure similar to the structural example illustrated in FIGS. 33 through 35 except for differences with the shapes of the blade edges of the blades.

The first forceps 2911 include a pair of blades 3301 and 3302. The blades 3301 and 3302 cross each other at an opening and closing shaft 2913 and can turn around the opening and closing shaft 2913. Moreover, parts of the blades 3301 and 3302 that extend toward the proximal end side beyond the opening and closing shaft 2913 make up a parallel crank 3303. Further, a linear-motion portion 3304 that is slidable back and forth in the longitudinal directions of the treatment unit 2910 is coupled to a proximal end of the parallel crank 3303.

Normally, the linear-motion portion 3304 is pushed in the direction of a distal end side (a far end side), under the returning force of a spring (not illustrated). Thus, the parallel crank 3303 has its lozenge shape opened, applying, in ganged relation thereto, a force tending to open the blades 3301 and 3302 around the opening and closing shaft 2913. Therefore, the first forceps 2911 are open as illustrated in FIG. 33 in the absence of a force applied thereto.

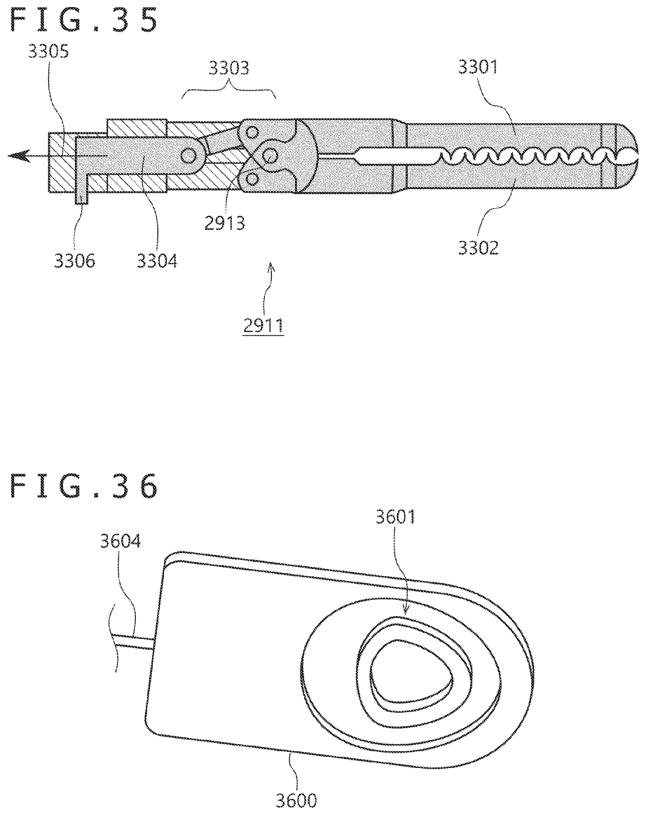

Further, when the linear-motion portion 3304 is pulled in the direction of the proximal end side (a near end side) indicated by the arrow denoted by the reference numeral 3305, the lozenge shape of the parallel crank 3303 is folded, applying, in ganged relation thereto, a force tending to close the blades 3301 and 3302 around the opening and closing shaft 2913. Thus, as illustrated in FIGS. 34 and 35, the first forceps 2911 are gradually closed depending on the distance that the linear-motion portion 3304 is pulled toward the proximal end side. Note that the linear-motion portion 3304 has a protrusion 3306 to be used when the linear-motion portion 3304 is to be pulled.

As can be recognized also from FIG. 29, the opening and closing shaft 2913 of the first forceps 2911 is perpendicular to a rotational axis of the switching shaft 2930. Thus, the first forceps 2911 have their blades openable and closable in directions parallel to the rotational axis of the switching shaft 2930. Stated otherwise, the first forceps 2911 are openable and closable in directions perpendicular to the path followed by the treatment unit 2910 when the treatment unit 2910 turns around the switching shaft 2930. This similarly applies to the second forceps 2912.

The storage portion 2940 is of a U shape having an opening along the path followed by a distal end of the treatment unit 2910. When the blades of the first forceps 2911 and the second forceps 2912 are closed upon transition from the usable state to the standby state, the first forceps 2911 and the second forceps 2912 can be placed into the U shape of the storage portion 2940. Moreover, once stored in the storage portion 2940, as the blades are kept within the U shape, they cannot be opened and closed. According to the example illustrated in FIG. 29, the first forceps 2911 that are in the usable state stay outside of the storage portion 2940 and have their blades openable and closable. On the other hand, since the second forceps 2912 that are in the standby state are stored in the storage portion 2940, their blades remain closed and cannot be opened.

The surgical instrument 2900 includes an opening and closing mechanism for opening and closing those of the first forceps 2911 and the second forceps 2912 that are in the usable state. The opening and closing mechanism also makes a closing motion with the blades when the first forceps 2911 and the second forceps 2912 transition from the usable state to the standby state. This is because the blades would impinge upon the U-shaped wall of the storage portion 2940 if they remain opened.

A structure and operation of the opening and closing mechanism included in the surgical instrument 2900 will be described below with reference to FIGS. 36 through 39.

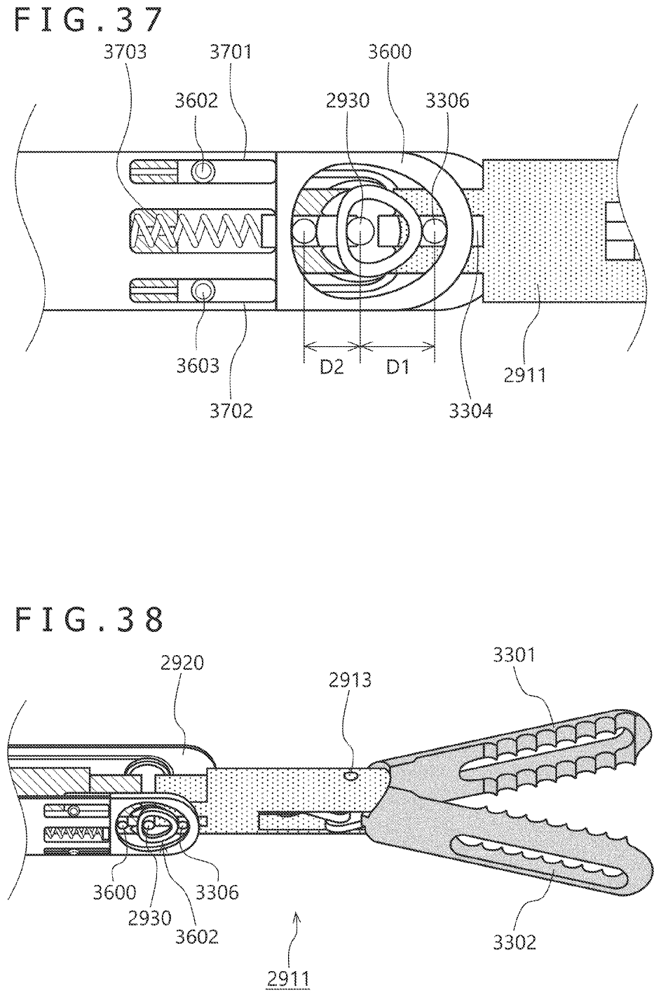

FIG. 36 illustrates in perspective a cam slider 3600 as a major component of the opening and closing mechanism. Further, FIG. 37 illustrates the cam slider 3600 as incorporated in the vicinity of the switching shaft 2930 of the surgical instrument 2900. In FIG. 37, however, a distal end portion of the holder 2920 is partly cut away to make the cam slider 3600 visible. The cam slider 2600 has a function of closing the blades while the treatment unit 2910 turns 180 degrees around the switching shaft 2930 as well as the function of opening and closing the forceps in the usable state.

As can be recognized from FIG. 36, the cam slider 3600 includes a recess 3601 defined therein by an inner wall surface that acts as a cam surface. In FIG. 37, the cam slider 3600 is partly cut off in the vicinity of the recess 3601, illustrating an opening for the viewer to understand parts in action within the recess 3601. As can be understood also from FIG. 37, the protrusion 3306 of the linear-motion portion 3304 for opening and closing the first forceps 2911 is accommodated in the recess 3601 and has its range of movement limited by the inner wall surface of the recess 3601. Further, a protrusion of a linear-motion portion for opening and closing the second forceps 2912 is similarly accommodated in the recess 3601 and has its range of movement limited by the inner wall surface of the recess 3601.

The inner wall surface of the recess 3601 in the cam slider 3600 has a cam profile whose center is disposed eccentrically closer to the forceps in the standby state (the near end side) than the center of rotation of the switching shaft 2930. In other words, the inner wall surface has a cam profile such that it has a longest radius from the switching shaft 2930 in the direction of the distal end side (the far end side) and a shortest radius from the switching shaft 2930 in the direction of the proximal end side (the near end side).

As illustrated in FIG. 37, when the first forceps 2911 are in the usable state and the second forceps 2912 are in the standby state, the distance D2 from the protrusion on the second forceps 2912 side to the center of rotation of the switching shaft 2930 is shorter than the distance D1 from the protrusion 3603 on the first forceps 2911 side to the center of rotation of the switching shaft 2930. Thus, since the linear-motion portion 3304 of the first forceps 2911 that are in the usable state is not pulled toward the proximal end side, the first forceps 2911 can be opened and closed. On the other hand, the linear-motion portion of the second forceps 2912 that are in the standby state is pulled in the direction opposite the blades by the protrusion held in abutment against the inner wall surface of the recess 3601. As a result, the blades of the second forceps 2912 in the standby state can be closed.

As illustrated in FIG. 37, a pair of protrusions 3602 and 3603 are disposed on an outer surface of the cam slider 3600 in an array perpendicular to the longitudinal directions of the treatment unit 2910. Moreover, the treatment unit 2910 has a pair of parallel guide grooves 3701 and 3702 defined linearly therein in the longitudinal directions thereof, with the protrusions 3602 and 3603 inserted respectively in the guide grooves 3701 and 3702. Thus, as the protrusions 3602 and 3603 slide respectively in the guide grooves 3701 and 3702, the cam slider 3600 slides in the longitudinal directions of the treatment unit 2910. Further, the range of movement of the cam slider 3600 is limited to the length of the guide grooves 3701 and 3702 or less.

Further, as illustrated in FIG. 37, the holder 2920 and the cam slider 3600 are coupled to each other by a helical spring 3703 that applies a force to the cam slider 3600, tending to push the cam slider 3600 toward the distal end side (the far end side). On the other hand, a wire 3604 for being pulled is attached to an end face of the cam slider 3600. When the wire 3604 is pulled, the cam slider 3600 is pulled back toward the proximal end side within a predetermined range of movement against the returning force of the helical spring 3703.

The forceps can be opened and closed by sliding the cam slider 3600 in the longitudinal directions of the cam slider 3600. Operation to open and close the first forceps 2911 by sliding the cam slider 3600 will be described below with reference to FIGS. 37 through 40.

FIGS. 37 and 38 illustrate the relative position of the cam slider 3600 and the opened/closed state of the first forceps 2911 at the time the helical spring 3703 acts on the cam slider 3600 but the cam slider 3600 is not pulled by the wire 3604. The cam slider 3600 is pushed in the direction of the distal end side (the far end side). Thus, since the protrusion 3306 can move in the direction of the distal end side (the far end side), the parallel crank 3303 has its lozenge shape opened, applying, in ganged relation thereto, a force tending to open the blades 3301 and 3302 around the opening and closing shaft 2913. As a result, the first forceps 2911 are open, as illustrated in FIG. 38.

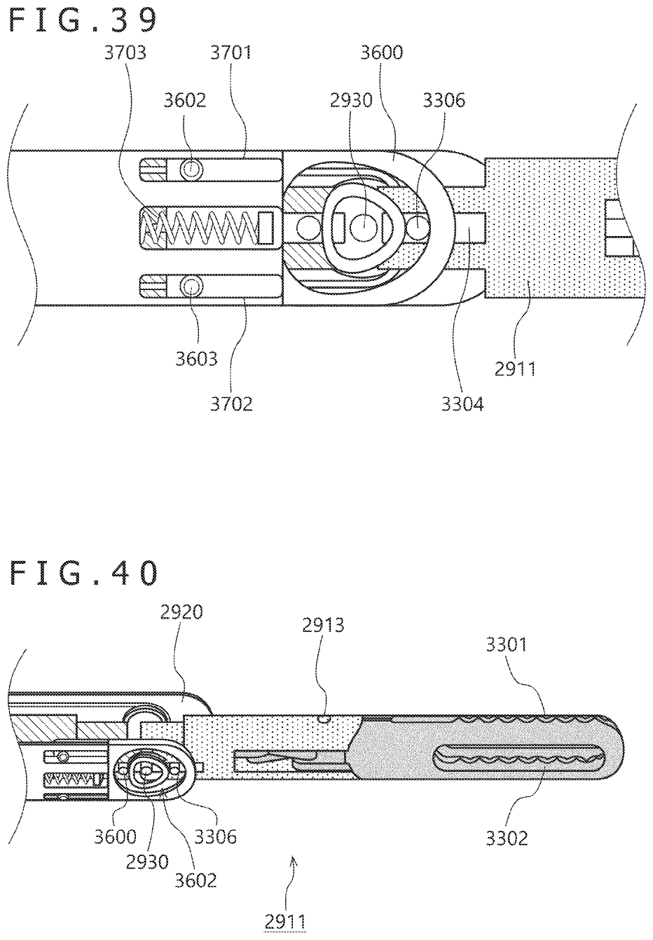

When the cam slider 3600 is pulled by the wire 3604 to slide toward the proximal end side (the near end side), the protrusion 3306 held in abutment against the inner wall surface of the recess 3601 moves toward the proximal end side (the near end side), as illustrated in FIG. 39, pulling the linear-motion portion 3304. As a result, the lozenge shape of the parallel crank 3303 is folded, closing the first forceps 2911 in ganged relation thereto, as illustrated in FIG. 40. Note that, although not illustrated and described in detail, also in a case where the second forceps 2912 are in the usable state, they can be opened and closed by similarly sliding the cam slider 3600.

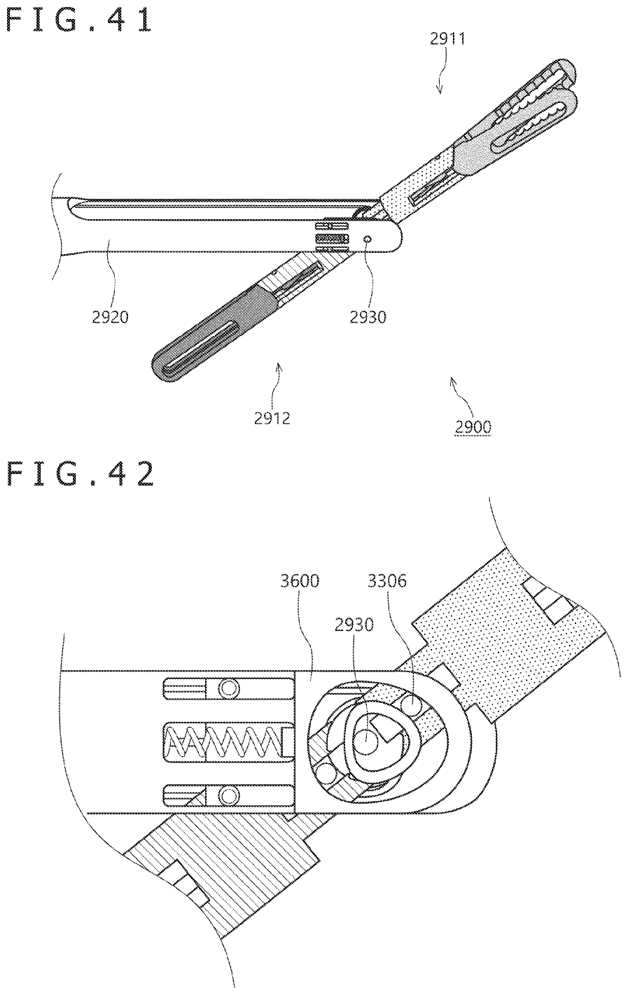

Next, operation to close the blades with the cam slider 3600 at the time the first forceps 711 transition from the usable state to the standby state will be described below further with reference to FIGS. 41 through 48. Note that, although not illustrated and described in detail, when the second forceps 2912 transition from the usable state to the standby state, their blades can also be closed by the cam slider 3600.

The first forceps 2911 as they are in the usable state are illustrated in FIGS. 37 and 38. The inner wall surface of the recess 3601 has a cam profile such that it has a longest radius from the switching shaft 2930 in the direction of the distal end side (the far end side), and a shortest radius from the switching shaft 2930 in the direction of the proximal end side (the near end side). On the first forceps 2911 that are in the usable state, therefore, the linear-motion portion 3304 moves toward the far end side, as can be recognized from FIG. 37, and the blades are open maximally, as illustrated in FIG. 38.

FIG. 41 illustrates the manner in which the treatment unit 2910 has turned approximately 45 degrees around the switching shaft 2930 counterclockwise in the plane of the sheet of FIG. 41 from the usable state of the first forceps 2911 (see FIG. 38). Further, FIG. 42 illustrates at an enlarged scale the cam slider 3600 and nearby parts in the position to which the treatment unit 2910 has turned. While the treatment unit 2910 is being turned, the protrusion 3306 is shifted in the direction of the switching shaft 2930 while sliding on the inner wall surface of the cam slider 3600. As a result, as can be recognized from FIG. 41, the blades of the first forceps 2911 start being closed.

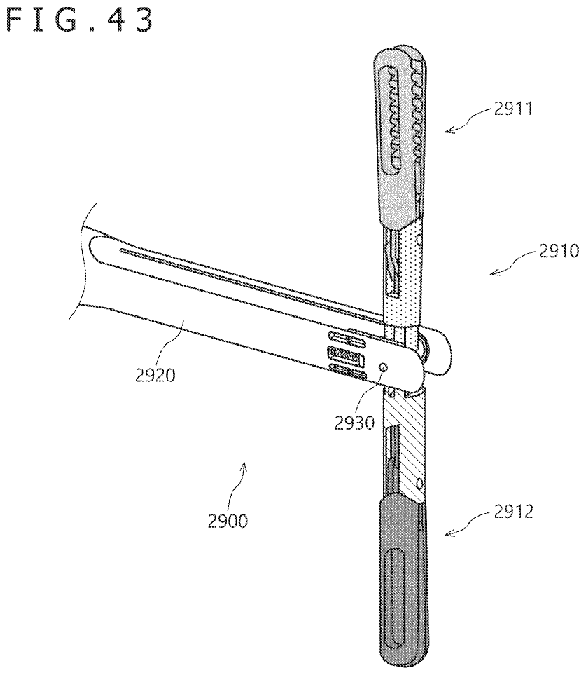

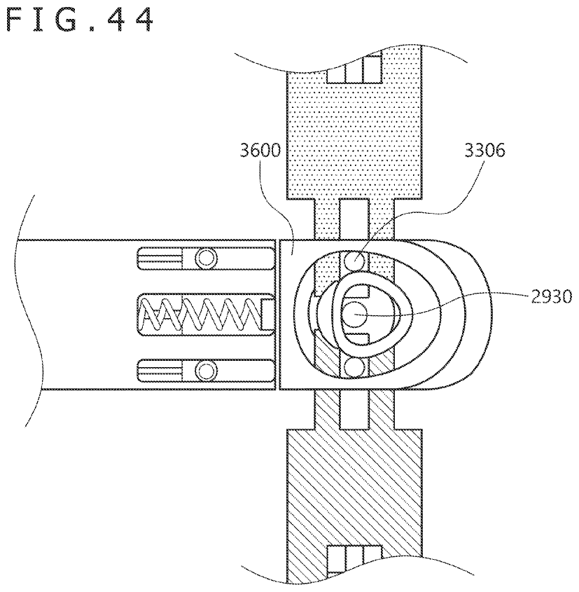

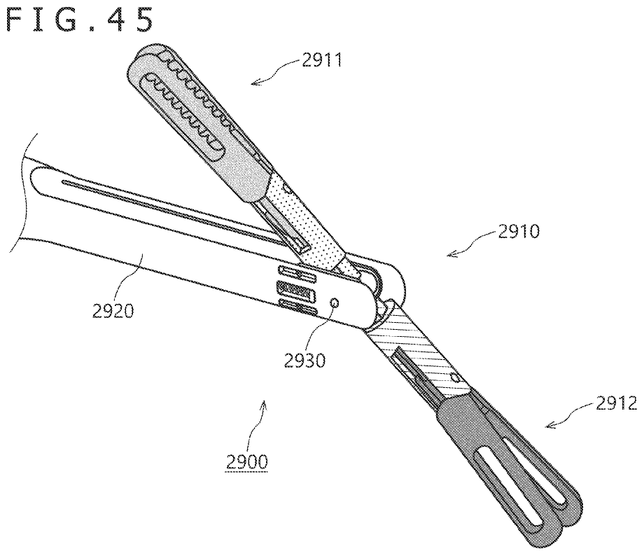

FIG. 43 illustrates the manner in which the treatment unit 2910 has turned approximately 90 degrees around the switching shaft 2930 counterclockwise in the plane of the sheet of FIG. 43 from the usable state of the first forceps 2911 (see FIG. 38). Further, FIG. 44 illustrates at an enlarged scale the cam slider 3600 and nearby parts in the position to which the treatment unit 2910 has turned. While the treatment unit 2910 is being further turned, the protrusion 3306 is further shifted in the direction of the switching shaft 2930 while sliding on the inner wall surface of the cam slider 3600. As a result, as can be recognized from FIG. 43, the blades of the first forceps 2911 are considerably closed.

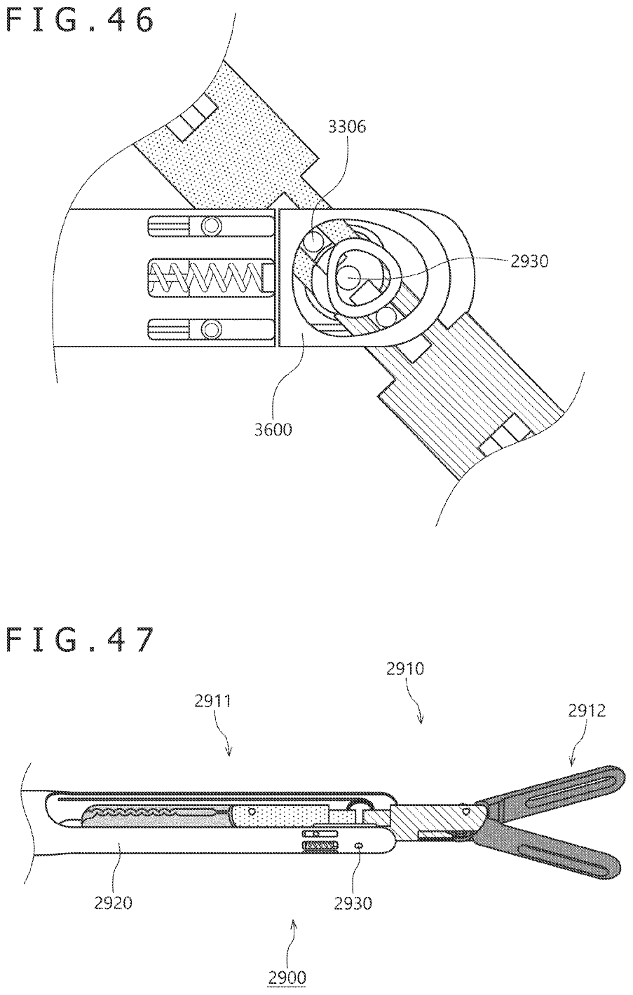

FIG. 45 illustrates the manner in which the treatment unit 2910 has turned approximately 135 degrees around the switching shaft 2930 counterclockwise in the plane of the sheet of FIG. 45 from the usable state of the first forceps 2911 (see FIG. 38). Further, FIG. 46 illustrates at an enlarged scale the cam slider 3600 and nearby parts in the position to which the treatment unit 2910 has turned. While the treatment unit 2910 is being further turned, the protrusion 3306 is further shifted in the direction of the switching shaft 2930 while sliding on the inner wall surface of the cam slider 3600. As a result, as can be recognized from FIG. 45, the blades of the first forceps 2911 are almost completely closed. On the contrary, the blades of the second forceps 2912 are considerably open.

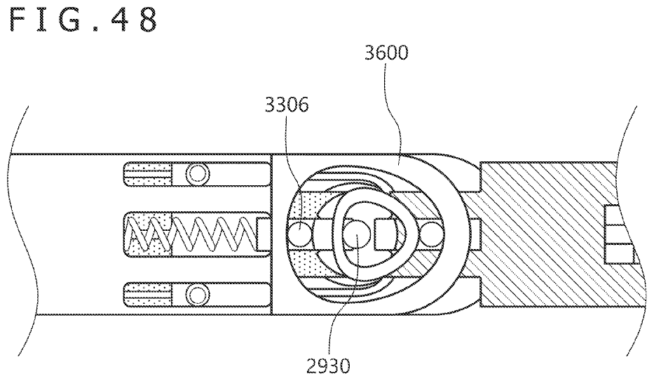

FIG. 47 illustrates the manner in which the treatment unit 2910 has turned approximately 180 degrees around the switching shaft 2930 counterclockwise in the plane of the sheet of FIG. 47 from the usable state of the first forceps 2911 (see FIG. 38). At this time, the first forceps 2911 are fully switched into the standby state, and the second forceps 2912 are fully switched into the usable state. Further, FIG. 48 illustrates at an enlarged scale the cam slider 3600 and nearby parts in the position to which the treatment unit 2910 has turned. While the treatment unit 2910 is being further turned, the protrusion 3306 is maximally shifted in the direction of the switching shaft 2930 while sliding on the inner wall surface of the cam slider 3600. As a result, as can be recognized from FIG. 47, the blades of the first forceps 2911 are completely closed and stored in the storage portion 2940. On the contrary, the blades of the second forceps 2912 are completely open.

Incidentally, the surgical instrument should preferably include for its usage a mechanism such as a latch, (not illustrated), for holding the treatment unit 2910 against rotation around the switching shaft 2930 at an angular position where one pair of the first forceps 2911 and the second forceps 2912 is in the usable state whereas the other pair is in the standby state (or when either the first forceps 2911 and the second forceps 2912 are in the usable state).

Further, in each of FIGS. 29 through 48, the holder 2920 has its proximal portion beyond the left end omitted from illustration because of the limited space available. It is assumed that the proximal end portion of the holder 2920 is of a shape and structure on which the slave arm of a surgical master-slave system, for example, can be mounted. Alternatively, the proximal end portion of the holder 2920 may be of a shape and structure that can be attached to manual forceps that are manually operable.

When the states of the first forceps 2911 and the second forceps 2912 are successfully switched, the successful switching may be indicated to the user (the surgeon or the like). For example, an encoder for detecting the angular position of the treatment unit 710 around the switching shaft 2930, another sensor, or the like may be used to detect when either the first forceps 2911 or the second forceps 2912 have been set in the usable state. Further, the detected result is output to the master side of the surgical master-slave system, and the master device indicates to the user that the switching of the treatment portion has been completed, by way of illumination (e.g., LED (Light Emitting Diode) blinking), sound (e.g., beeping), haptic technology, monitor screen display, or the like.

The surgical instrument 2900 according to the third embodiment is used in laparoscopic surgery, for example, by being inserted through a trocar into a body cavity of the patient. For example, the surgeon may confirm a space in the body cavity large enough to turn the treatment unit 2910 with safety therein and may then switch the forceps in the body cavity without removing the surgical instrument 2900 from the body cavity. Alternatively, the surgical instrument 2900 may incorporate a distance sensor (a TOF distance sensor, a laser distance sensor, an IR distance sensor, or the like), and the surgeon may measure the distance up to surrounding tissues with the distance sensor, confirm a space in the body cavity large enough to turn the treatment unit 2910 with safety therein, and then switch the forceps in the body cavity without removing the surgical instrument 2900 from the body cavity.

The surgical instrument 2900 according to the third embodiment can be used in two ways with the first forceps 2911 and the second forceps 2912 by turning the treatment unit 2910 through 180 degrees (or stated otherwise, without removing the surgical instrument 2900 from the body cavity and changing the forceps as in the conventional art). Thus, a plurality of forceps can be used on the same surgical instrument in a space saving environment. Further, the treatment instruments can be switched around in a short period of time by simply turning the treatment unit 2910. Consequently, the time required for the surgical operation is reduced by reducing the time required for switching the surgical instrument, resulting in a commensurate reduction in the burden on the patient.

In a case where a surgical operation is carried out using the surgical instrument 2900 according to the second embodiment mounted on a surgical robot, the number of surgical instruments to be installed on the surgical robot can be reduced, or, in a case where the same number of surgical instruments as in the past are installed on the surgical robot, more treatment instruments can simultaneously be used. Further, if the surgical instrument 2900 is sterilized or cleaned, then the two kinds of treatment instruments are simultaneously sterilized or cleaned. In addition, as the number of times that surgical instruments on the surgical robot are replaced is reduced, the time required to change steps of the surgical operation is shortened, reducing the trouble that the surgeon has to take.

### Embodiment 4

According to a fourth embodiment, a surgical master-slave robot system that uses a surgical instrument capable of switching the states of use of two kinds of treatment instruments will be described below. However, a surgical instrument to be attached as an end effector to a slave device may be the surgical instrument according to any one of the first through third embodiments described above.

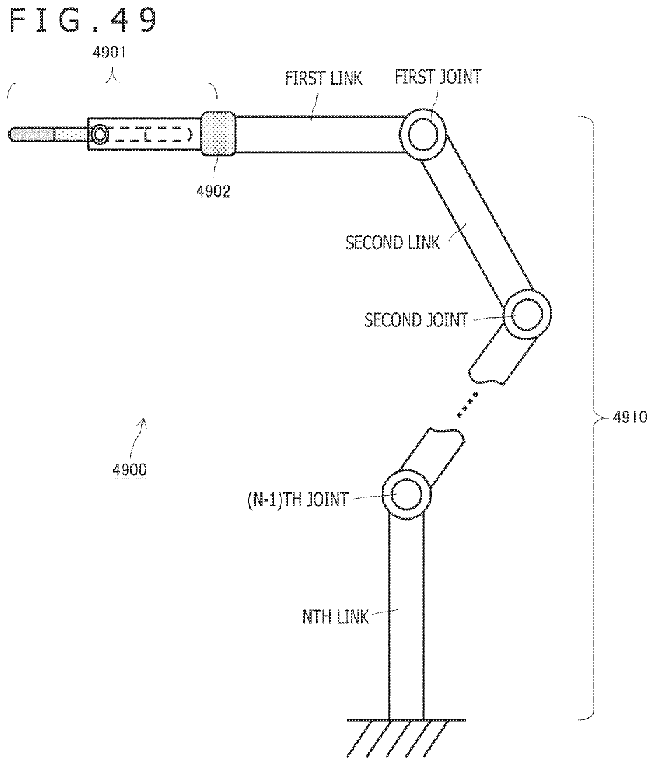

FIG. 49 schematically illustrates a structural example of a multi-joint arm 4900. The illustrated multi-joint arm 4900 is used in laparoscopic surgery. It is used in laparoscopic surgery. The multi-joint arm 4900 is, for example, applied as a slave arm of a master-slave robot system, and is controlled to reflect motion of a master arm that is remotely operated by a person (the surgeon).

The multi-joint arm 4900 includes an end effector 4901 and a multi-joint link structural body 4910 having a distal end to which the end effector 4901 is attached through a predetermined attachment 4902.

Specifically, the end effector 4901 includes a treatment instrument to be inserted and used in a body cavity of a patient in laparoscopic surgery, and is inserted through an artificial hole such as a trocar or a natural hole such as a nostril or mouth into the body cavity of the patient. The treatment instrument may be forceps, an aeroperitoneum tube, an energy treatment instrument, tweezers, a retractor, or the like, for example. Alternatively, the end effector may be an endoscope or an exoscope for observing the surgical site. A camera head with a scope (a direct-view scope or an oblique-view scope) mounted thereon captures an image of the surgical field, and the observing direction can be changed by controlling the joints of the multi-joint arm 4900. In FIG. 49, only one end effector 4901 is illustrated. However, the end effector 4901 can be replaced with many kinds of end effectors that are compatible with the attachment 4902.

Structural details of the multi-joint link structural body 4910 that include the number of shafts (the number of joints), the degrees of freedom of the shafts, the number of links (the number of arms, etc. are optional. Hereinafter, the links included in the multi-joint link structural body 4910 will be referred to as a first link, a second link, and so on successively from the far end side (the rear end of the end effector 4901), for convenience of description. Further, the joints included in the multi-joint link structural body 4910 will be referred to as a first joint, a second joint, and so on successively from the far end side (the rear end of the end effector 4901).

Either one of the surgical instruments according to the first through third embodiments described above may be used as one of many kinds of end effectors 4901 attached to the distal end of the multi-joint link structural body 4910 by the attachment 4902.

For example, the surgical instrument 100 according to the first embodiment includes the treatment unit 110 that has the first treatment portion 111 and the second treatment portion 112 respectively on its both ends and that is held by the holder 120. One of the first treatment portion 111 and the second treatment portion 112 can selectively be used by turning the treatment unit 110 about the switching shaft 130 through 180 degrees. Thus, a plurality of treatment instruments can be used on the same surgical instrument in a space saving environment. Further, the treatment instruments can be switched around in a short period of time by simply turning the treatment unit 110.

Further, it is possible to construct a surgical robot system including a plurality of multi-joint arms combined with each other as illustrated in FIG. 49. By applying the surgical instruments disclosed in the present description to at least some arms of the surgical robot of this kind, there are provided advantages in that the total number of surgical instruments to be attached can be reduced, or, in a case where the same number of surgical instruments as in the past are installed on the surgical robot, more treatment instruments can simultaneously be used. Further, if the surgical instrument is sterilized or cleaned, then the two kinds of treatment instruments are simultaneously sterilized or cleaned. In addition, as the number of times that surgical instruments on the surgical robot are replaced is reduced, the time required to change steps of the surgical operation is shortened, reducing the trouble that the surgeon has to take.

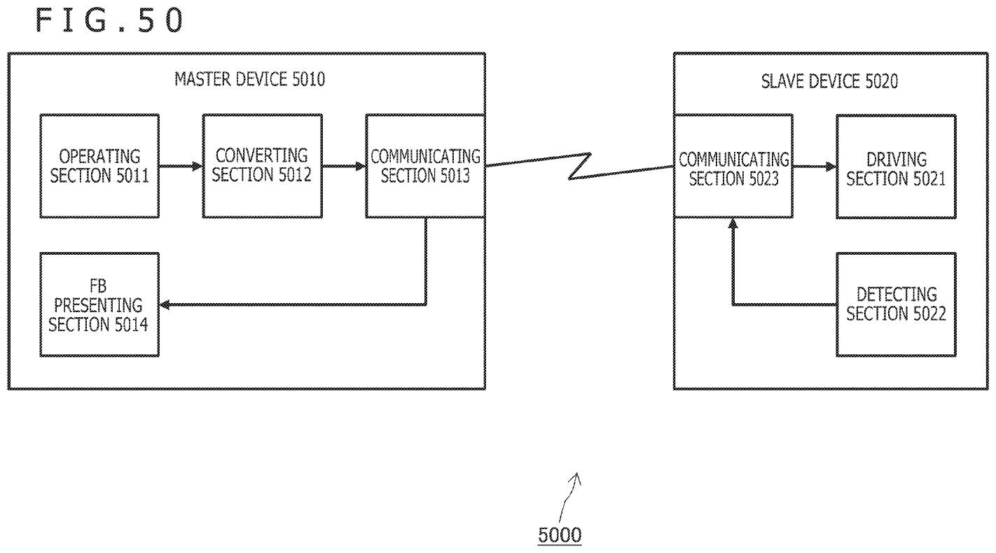

FIG. 50 schematically illustrates a functional configuration of a master-slave surgical system 5000. It is assumed that the multi-joint arm 4900 illustrated in FIG. 49 is used as one of slave arms. The surgical system 5000 includes a master device 5010 operable by the operator and a slave device 5020 remotely controllable from the master device 5010 by the operator. The master device 5010 and the slave device 5020 are connected to each other by a wireless or wired network.

The master device 5010 includes an operating section 5011, a converting section 5012, a communicating section 5013, and a feedback (FB) presenting section 5014.

The operating section 5011 includes a master arm or the like used by the operator to remotely control the slave device 5020. The converting section 5012 converts the content of an operation that the operator has performed on the operating section 5011 into control information for controlling the actuation of the slave device 5020 (or, more specifically, a driving section 5021 of the slave device 5020). In addition, in a case where the surgical instrument according to either one of the first through third embodiments is used as an end effector on a slave arm of the slave device 5020, the operator is able to instruct the slave device 5020 to switch the treatment instruments by turning the treatment unit with the operating section 5011, for example.

The communicating section 5013 is connected to the slave device 5020 (or, more specifically, a communicating section 5023 of the slave device 5020), by the wireless or wired network. The communicating section 5013 sends control information output from the converting section 5012 to the slave device 5020.

The slave device 5020 includes the driving section 5021, a detecting section 5022, and the communicating section 5023.

It is assumed that the slave device 5020 includes, as a slave arm, the multi-joint arm 4900 with a surgical instrument attached as an end effector thereto as illustrated in FIG. 49. The driving section 5021 includes an actuator for turning the joints that interconnect the links of the multi-joint arm 4900 and an actuator for operating the end effector (e.g., opening and closing forceps).

The detecting section 5022 includes a sensor for detecting a physical action (e.g., an external force, a temperature, or the like) that the end effector such as forceps receives from a tissue.

The communicating section 5023 is connected to the master device 5010 (more specifically, the communicating section 5013 of the master device 5010) by the wireless or wired network. The driving section 5021 is actuated according to control information that the communicating section 5023 has received from the master device 5010. In a case where the surgical instrument according to either one of the first through third embodiments is used as the end effector on the slave arm, the driving section 5021 switches the treatment instruments by turning the treatment unit. In addition, detected results from the detecting section 5022 are sent from the communicating section 5023 to the master device 5010.

In the master device 5010, the feedback (FB) presenting section 5014 performs the presentation of feedback to the operator on the basis of the detected results (the external force, the temperature, or the like) that the communicating section 5013 has received from the slave device 5020 as feedback information.

The operator who is operating the master device 5010 can recognize through the feedback presenting section 5014 a physical action (a reactive force received from a tissue, a body temperature of a tissue, or the like) that is applied to the end effector of the slave device 5020. For example, if the slave device 5020 is a surgical robot, then the operator can feel a response applied to the treatment instrument such as forceps by way of haptic feedback, and thus can appropriately make manual adjustments in forming stitches and completing sutures, thereby efficiently operating on the patient while making the surgical operation minimally invasive to the biotissue.

### INDUSTRIAL APPLICABILITY

The technology disclosed in the present description has been described in detail above with reference to the particular embodiments. However, it is obvious that those skilled in the art can make modifications and substitutions in the embodiments without departing from the principles of the technology disclosed in the present description.

The surgical instruments disclosed in the present description can be attached to the slave arm of a surgical master-slave system or manual forceps, for example, to use two types of treatment instruments simultaneously in a space saving environment. Further, the treatment instruments can be switched around in a short period of time by simply turning the treatment unit.

In a case where a surgical operation is carried out using the surgical instruments disclosed in the present description mounted on a surgical master-slave system, the number of surgical instruments to be installed on the surgical master-slave system can be reduced, or, in a case where the same number of surgical master-slave system as in the past are installed on the surgical robot, more treatment instruments can simultaneously be used. Further, if the surgical instrument is sterilized or cleaned, then the two kinds of treatment instruments are simultaneously sterilized or cleaned. In addition, as the number of times that surgical instruments on the surgical robot are replaced is reduced, the time required for changing steps of the surgical operation is shortened, reducing the trouble that the surgeon has to take.

Put otherwise, the technology disclosed in the present description has been described by way of illustrative example, and the contents of the present description should not be construed according to restrictive interpretation. The scope of claims for patent should be taken into account for determining the principles of the technology disclosed in the present description.

Note that the technology disclosed in the present description can also take the following arrangements

(1) A surgical instrument including:

a treatment unit having a first treatment portion on an end portion thereof and a second treatment portion on another end portion thereof; and

a holder that supports the treatment unit so as to allow states of use of the first treatment portion and the second treatment portion to be switched.

(1-1) The surgical instrument according to (1) described above, in which each of the first treatment portion and the second treatment portion includes any one of forceps, an aeroperitoneum tube, an energy treatment instrument, tweezers, and a retractor.

(2) The surgical instrument according to (1) described above, in which the holder supports the holder so as to be rotatable around a switching shaft for switching the states of use of the first treatment portion and the second treatment portion.

(3) The surgical instrument according to (1) described above, in which the holder supports the treatment unit such that, while one of the first treatment portion and the second treatment portion is in a usable state, the other is in a standby state.

(4) The surgical instrument according to (3) described above, in which the holder includes a storage portion that stores one of the first treatment portion and the second treatment portion that is in the standby state.

(4-1) The surgical instrument according to (3) described above, in which one of treatment portions that is inserted in the storage portion is in the standby state, and the other that emerges out of the storage portion is in the usable state.

(5) The surgical instrument according to (4) described above, in which the holder supports the treatment unit in a manner allowing the treatment unit to be rotatable around a switching shaft, and

the treatment unit is rotated around the switching shaft to switch the first treatment portion and the second treatment portion alternately between the usable state and the standby state.

(6) The surgical instrument according to (2), further including:

a switching mechanism that turns the treatment unit around the switching shaft.

(7) The surgical instrument according to any one of (3) through (6) described above, in which at least one of the first treatment portion and the second treatment portion includes an openable and closable end effector, and

the storage portion stores the openable and closable end effector therein in such a manner that the openable and closable end effector remains closed when the openable and closable end effector is in the standby state.

(8) The surgical instrument according to (7) described above, in which the storage portion has a cross-sectional shape having an opening along a direction around the switching shaft, and

the openable and closable end effector is openable and closable around an opening and closing shaft perpendicular to the switching shaft.

(9) The surgical instrument according to (8) described above, further including:

an opening and closing mechanism that opens and closes the openable and closable end effector.

(10) The surgical instrument according to (9) described above, in which the opening and closing mechanism closes the openable and closable end effector when the openable and closable end effector transitions to the standby state.

(11) The surgical instrument according to (10) described above, in which the opening and closing mechanism closes the openable and closable end effector in ganged relation to turning of the treatment unit around the switching shaft.

(12) A surgical system including:

a surgical instrument that includes a treatment unit having a first treatment portion on an end portion thereof and a second treatment portion on another end portion thereof, and a holder that supports the treatment unit so as to allow states of use of the first treatment portion and the second treatment portion to be switched;

an arm with the surgical instrument attached thereto, the arm including at least one joint; and

a master device that outputs a signal for actuating the joint of the slave device.

(13) The medical manipulator system according to (12) described above, further including:

a master device that outputs a signal for actuating the joint of the arm and a movable portion of the surgical instrument.

### REFERENCE SIGNS LIST

100: Surgical instrument (first embodiment)
110: Treatment unit
111: First treatment portion
112: Second treatment portion
120: Holder
130: Switching shaft
140: Storage portion
700: Surgical instrument (second embodiment)
710: Treatment unit
711: First forceps
712: Second forceps
713: Opening and closing shaft (for first forceps)
714: Opening and closing shaft (for second forceps)
720: Holder
730: Switching shaft
740: Storage portion
750: Switching mechanism
760: Opening and closing mechanism
1301: Pulley
1302: Wire
1501, 1502: Blade
1503: Parallel crank
1504: Linear-motion portion
1701: Wire
1901: Insertion hole
2100: Cam slider
2101: Opening
2111: Protrusion (first forceps 711 side)
2112: Protrusion (second forceps 712 side)
2121: Wire
2900: Surgical instrument (third embodiment)
2910: Treatment unit
2911: First forceps
2912: Second forceps
2913: Opening and closing shaft (for first forceps)
2914: Opening and closing shaft (for second forceps)
2920: Holder
2930: Switching shaft
2940: Storage portion
3301, 3302: Blade
3303: Parallel crank
3304: Linear-motion portion
3306: Protrusion
3600: Cam slider
3601: Recess
3602, 3603: Protrusion
3701, 3702: Guide groove
3703: Helical spring
4900: Multi-joint arm
4901: End effector
4902: Attachment
4910: Multi-joint link structural body
5000: Surgical system
5010: Master device
5011: Operating section
5012: Converting section
5013: Communicating section
5014: Feedback presenting section
5020: Slave device
5021: Driving section
5022: Detecting section
5023: Communicating section

1. A surgical instrument comprising:
a treatment unit having a first treatment portion on an end portion thereof and a second treatment portion on another end portion thereof; and
a holder that supports the treatment unit so as to allow states of use of the first treatment portion and the second treatment portion to be switched.

2. The surgical instrument according to claim 1, wherein the holder supports the holder so as to be rotatable around a switching shaft for switching the states of use of the first treatment portion and the second treatment portion.

3. The surgical instrument according to claim 1, wherein the holder supports the treatment unit such that, while one of the first treatment portion and the second treatment portion is in a usable state, the other is in a standby state.

4. The surgical instrument according to claim 3, wherein the holder includes a storage portion that stores one of the first treatment portion and the second treatment portion that is in the standby state.

5. The surgical instrument according to claim 4, wherein the holder supports the treatment unit in a manner allowing the treatment unit to be rotatable around a switching shaft, and
the treatment unit is rotated around the switching shaft to switch the first treatment portion and the second treatment portion alternately between the usable state and the standby state.

6. The surgical instrument according to claim 2, further comprising:
a turning mechanism that turns the treatment unit around the switching shaft.

7. The surgical instrument according to claim 3, wherein at least one of the first treatment portion and the second treatment portion includes an openable and closable end effector, and
the storage portion stores the openable and closable end effector therein in such a manner that the openable and closable end effector remains closed when the openable and closable end effector is in the standby state.

8. The surgical instrument according to claim 7, wherein the storage portion has a cross-sectional shape having an opening along a direction around the switching shaft, and
the openable and closable end effector is openable and closable around an opening and closing shaft perpendicular to the switching shaft.

9. The surgical instrument according to claim 8, further comprising:
an opening and closing mechanism that opens and closes the openable and closable end effector.

10. The surgical instrument according to claim 9, wherein the opening and closing mechanism closes the openable and closable end effector when the openable and closable end effector transitions to the standby state.

11. The surgical instrument according to claim 10, wherein the opening and closing mechanism closes the openable and closable end effector in ganged relation to turning of the treatment unit around the switching shaft.

12. A medical manipulator system comprising:
a surgical instrument that includes a treatment unit having a first treatment portion on an end portion thereof and a second treatment portion on another end portion thereof, and a holder that supports the treatment unit so as to allow states of use of the first treatment portion and the second treatment portion to be switched; and
an arm with the surgical instrument attached thereto, the arm including at least one joint.

13. The medical manipulator system according to claim 12, further comprising:
a master device that outputs a signal for actuating the joint of the arm and a movable portion of the surgical instrument.
